(19) United States
(12) Patent Application Publication
(10) Pub. No.: US 2018/0263595 A1
(43) Pub. Date: Sep. 20, 2018

(54) HAND-HELD MEDICAL APPARATUS AND MEDICAL ULTRASOUND SYSTEM

(71) Applicant: ETH ZURICH, Zürich (CH)

(72) Inventors: Orcun GOKSEL, Zürich (CH); Sergio SANABRIA, Zürich (CH)

(73) Assignee: ETH ZURICH, Zürich (CH)

(21) Appl. No.: 15/756,504

(22) PCT Filed: Aug. 29, 2016
(86) PCT No.: PCT/EP2016/070321

§ 371 Date: Feb. 28, 2018

(30) Foreign Application Priority Data
Aug. 28, 2015 (EP) 15182877.9

(51) Int. Cl. A61B 8/15; A61B 8/08; A61B 8/00

(52) U.S. Cl. CPC A61B 8/15; A61B 8/0825; A61B 8/483; A61B 8/5269; A61B 8/4209; A61B 8/587; G06T 11/005; A61B 8/4263; A61B 8/4427; A61B 8/4488; A61B 8/4254; A61B 8/5207; A61B 8/085

(57) ABSTRACT

A hand-held medical ultrasound apparatus (10) comprises an ultrasound transducer (1) for emitting ultrasound, a reflector (2) for reflecting at least a portion of the emitted ultrasound, and an indicator (21,22,311,312) enabling the indication of a relative position and/or orientation between the transducer (1) and the reflector (1).

### TECHNICAL FIELD

The invention is related to a hand-held medical ultrasound apparatus, and to a medical ultrasound system.

### BACKGROUND ART

Tumors and certain other anomalies in breast tissue are not always detectable in conventional B-mode ultrasound systems. However, these pathologies may present high contrast regarding other ultrasound characteristics, such as ultrasound propagation speed and attenuation. Similarly to X-ray Computed Tomography (CT), in order to obtain spatially-resolved images of these parameters, ultrasound waves are transmitted and recorded at multiple angular directions. This presently requires high-end application-specific Ultrasound Computed Tomography (USCT) equipment, based on a large number of stationary ultrasound sensors positioned around the breast, see “Breast density measurements with ultrasound tomography: A comparison with film and digital mammography”, Duric et al., Med. Phys. 40(1), January 2013, or by mechanically rotating ultrasound sensors around the breast, see “Imaging of Sound Speed Using Reflection Ultrasound Tomography”, Nebeker et al. Ultrasound Med 2012, 31, pages 1389-1404, both of which systems require the immersion of the breast in a water tank and the scanning with a bulky custom-made ultrasound system. Though accurate results, such systems are burdensome in daily clinical use, require additional space in the clinics and specialized personnel to perform, and are typically costly which can only be used for the given specific purpose.

Extensions of current X-ray mammography systems for ultrasound sound-speed and attenuation imaging have been investigated, for example see “Limited-angle ultrasonic transmission tomography of the compressed female breast. Krueger et al. IEEE Ultrasonics Symposium 1998, pages 1345-1348”. In this case, the breast is fully compressed between two stationary compression plates, and ultrasound transducers are positioned over and/or below the compression plates. In other embodiments, one of the plates is eliminated and the breast is compressed between a transducer and a stationary plate, see for example “Reconstruction of ultrasonic sound velocity and attenuation coefficient using linear arrays: clinical assessment. Chang et al. 2007 1681-1687”. The compression of the breast according to these setups results into a painful diagnosis procedure, similarly to X-ray mammography. It also reduces flexibility, since the ultrasound transducers are either fixed or restricted to move parallel to the compression plates, having only access to coronal planes. Small-sized breast and ultrasound imaging near the chest wall can also prove unfeasible. With respect to USCT equipment, the mammography setup allows only for transmitting and recording through a limited set of angular directions, which leads to strong artifacts in the obtained ultrasound images if the positions and geometry of the anomalies (e.g. tumors) are not known a priori. Moreover, small air gaps are difficult to avoid between the compression plates and the breast, and induce strong artifacts in the ultrasound images. As a consequence, the quality of the diagnoses obtained with these systems is presently poor and none have therefore reached to a commercial implementation level.

### DISCLOSURE OF THE INVENTION

The problem to be solved by the present invention is therefore to enable a widespread use of ultrasound computed tomography (USCT).

This problem is solved by a hand-held medical ultrasound apparatus, comprising an ultrasound transducer for emitting ultrasound, a reflector for reflecting at least a portion of the emitted ultrasound, and, preferably, an indicator enabling the indication of a relative position and/or orientation between the transducer and the reflector.

The apparatus not necessarily encompasses a full scale ultrasound computed tomography system, however, the apparatus can be part of and/or connected to such a tomographic unit of such system.

The ultrasound apparatus is a medical apparatus which implies that its use is in a medical context: The apparatus may be used for one or more of medical screening, diagnosis, staging (e.g. of cancer), preoperative planning, intra-operative guidance, and post-operative follow-up.

Hand-held in this context is meant to be portable, or mobile. The apparatus can be held by a sonographer such as a doctor or a nurse during inspecting a patient. Hence, its weight and its extension are dimensioned to apply the apparatus at any place without being bound to a stationary set-up for the transducer.

The ultrasound transducer comprises at least an element for emitting ultrasound, preferably at some frequency in a range between 1 MHz and 40 MHz, and more preferably in a range between 3 MHz and 14 MHz. The ultrasound transducer preferably converts electrical signals into ultrasound waves, e.g. by means of a piezoelectric converter as such element. In a very preferred embodiment, the ultrasound transducer also comprises at least one receiver element, and preferably more, for receiving ultrasound waves, and in particular for receiving reflected ultrasound waves as will be explained below, and for converting the received ultrasound waves into electrical signals.

The apparatus further comprises a reflector for reflecting ultrasound waves emitted by the transducer and travelling through the inspected tissue. Hence, the reflector is of ultrasound reflective property, which may be achieved by choosing the reflector at a different acoustic impedance than the tissue or by applying a material in or on the reflector that is reflective for ultrasound, such as metal (e.g. aluminium, steel), polymers/plastics (e.g. PMMA, Polycarbonate, ABS, rubber, silicone), entrapped air or fluid layers, glass, ceramics, mineral aggregates and other composites or metamaterials.

In operation, it is preferably envisaged that the body tissue to be examined, which preferably is the female breast, is arranged between the transducer and the reflector. It is preferred that the reflector and the transducer are arranged with respect to each other such that the reflector is exposed at least to a part of the ultrasound emitted by the transducer after travelling through the tissue. Preferably, the transducer and the reflector are arranged opposite to each other with the reflector facing directly to or roughly toward the transducer.

Ultrasound waves are sequentially transmitted from the one or more emitting elements, transmitted through the breast target, reflected and/or scattered at the reflector plate and re-acquired by one or more of the receiving elements. This allows for the measurement of ultrasound parameters, in particular ultrasound propagation speed and/or ultrasound attenuation along different angular directions, and allows for the reconstruction of an USCT image in a tomographic unit that the apparatus is connected to. The tomographic unit is understood to convert the signals provided by the apparatus into images to be displayed to the sonographer, for example.

The indicator of the apparatus, if any, enables the indication of a relative position and/or orientation between the transducer and the reflector. It is not required to always indicate both the position and the orientation, wherein the position preferably refers to a distance between the transducer and the reflector while the orientation refers to an angle between the transducer and the reflector. One of these two measures may be sufficient, in particular when e.g. the other measure is predefined anyway, e.g. by way of the arrangement of the transducer and the reflector in the apparatus. The indicator not necessarily needs to show the position and/or orientation at the apparatus itself; it may just enable so. In one embodiment, there are provided means at the apparatus itself to derive the positional and/or orientation information in an ad hoc manner by the user. Such means may preferably include a scale or other visual indicators allowing to assess e.g. a distance between the transducer and the reflector. In another embodiment, the apparatus may contain a sensor for one or more of determining the position and/or the orientation. Here, a corresponding sensor signal may be evaluated and the position and/or orientation may be determined in a remote unit such as a tomographic unit to which the sensor signal may be transmitted. In a third variant, the reflector itself may be prepared in a way to allow the identification of the reflector-transducer position/orientation in the image derived from the reflected ultrasound received by the transducer and finally displayed on a display of a tomographic unit.

It is preferred that the apparatus is used in Ultrasound Computed Tomography (USCT) in the medical domain to detect tumorous inclusions in breast tissue, which may not be visible in conventional B-mode images or may be visible but may not be diagnosed or categorized in B-mode images alone. Preferably, the apparatus is prepared to allow a measurement of the speed of ultrasound on its way from the transducer to the reflector and back to the transducer. By transmitting ultrasound waves through tissue between the ultrasound transducer and a reflector of known position and orientation and back through the tissue to the transducer, an USCT image can be obtained. An ultrasound parameter of the ultrasound wave can be computed dependent on the length of the path the ultrasound travels, which in the most simple case equals twice the distance between the transducer and the reflector, and dependent on the time taken for travelling this path, which is the time measured between emitting an ultrasound pulse and receiving a reflected portion of the ultrasound pulse. Hence, the present apparatus preferably can be considered as a handheld extension of an USCT tomographic unit.

Preferably, the ultrasound parameter that is determined per cell can be one of speed of (ultra)sound, acoustic attenuation, frequency dependent acoustic quantities, speed of sound dispersion. Although the following embodiments are mostly referred to the speed of sound determined as ultrasound parameter, it is understood that in any of the following embodiments, the speed of sound may be replaced by acoustic attenuation as relevant ultrasound parameter, or any of the other parameters as listed.

The measured ultrasound parameters can be in turn combined to estimate other tissue properties, such as for instance the tissue temperature (e.g., during an ablation treatment), or the mass density, or in general any property of healthy or diseased tissue, which correlates with the measured ultrasound parameters. Repeated ultrasound measurements can be used to monitor tissue changes in time.

The measured ultrasound parameters can as well be determined in function of an external perturbation applied to the tissue, such as a mechanical excitation (for instance, a pre-compression or a vibration field, such as vocal fremitus), or a temperature field (for instance, during an ablation treatment), among others.

Preferably, the present ultrasound system comprising the hand-held apparatus according to any of the embodiments and a processing unit for determining the tomographic image is embodied to identify ultrasound echos from the reflector, and detect perturbations in the relevant acoustic parameters, such as speed of sound or attenuation, introduced by the presence of tissue heterogeneities such as tumors.

For this purpose, the ultrasound transducer comprises a set of emitter elements and a set of receiver elements. While the elements of the sets may be different elements such that emitter elements only are capable of emitting ultrasound while receiver elements only are capable of receiving ultrasound, in a different embodiment a single transducer element may be configured to emit and receive ultrasound. Such transducer element is referred to act as emitter element and as receiver element respectively. Each set preferably comprises two or more elements, and preferably more than hundred elements.

Preferably, a combination of an emitter element and a receiver element—also referred to as pair—is operated at the same time, i.e. the processor triggers the respective emitter element to emit an ultrasound wave, while the receiver element receives the emitted and reflected ultrasound wave with a certain delay. On its trace, the ultrasound wave travels from the emitter element through tissue arranged between the transducer and the reflector, to the reflector and back through the tissue to the receiver element, thereby defining a ray path. At the receiver element, the received reflected ultrasound wave is converted into an electrical signal over time, also referred to as radio frequency (RF) trace.

Accordingly, the time delay is measured in form of a time difference between the time of emission of the ultrasound wave from the emitter element, and the time of receipt of the reflected ultrasound wave at the receiver element. This time delay is also referred to as time of flight. Given that various emitter-receiver element combinations are triggered sequentially by the processor, it is preferred that for each combination the corresponding RF trace is recorded. Preferably, all possible emitter element-receiver element combinations are triggered and define the set of combinations. However, in a different embodiment, only a selection out of all possible combinations is defined in the set of combinations.

In a preferred embodiment, a single transducer with N transducer elements is applied, and a “multi-static matrix”, with RF traces, and/or corresponding time of flight values for all possible N×N emitter element-receiver element combinations is recorded. It is then preferred, that for identifying a certain path p, an index for the transducer emitter element e and the transducer receiver element r are used, so that the time of flight tₚ and t_{e,r} are equivalent.

Other variations are possible: For instance, several adjacent emitter elements may be fired simultaneously or with an incremental time-delay, generating a so-called “plane-wave” emission, to increase an acoustic intensity level coupled into the measured tissue, and/or the RF traces of several adjacent transducers may be averaged or combined in any form to reduce noise. Hence, the preferred starting point to the image reconstruction is a set of digitized RF traces acquired by individual receiver elements upon specific emitter firings, hence, each corresponding to an emitter element-receiver element position pair. From this RF trace matrix, a corresponding time of flight matrix tₚ may be generated, e.g. by analyzing the RF traces. This processing step is also referred to as delineation.

The ray path p is assumed to be in the plane defined by the transducer and the reflector. The plane preferably is discretized into cells traversed by a finite set of ray paths p corresponding to different emitter element-receiver element pairs. In operation, when tissue is arranged between the transducer and the reflector, these cells reflect locations in the tissue in the subject plane. This cell structure supports the localization of portions of tissue that may be considered as tumorous, which portions are also referred to inclusions. The cell size is to be defined upfront and determines the resolution of the image. The process of determining the ultrasound parameter per cell based on the time of flight values is also referred to as reconstruction. Finally, the processor is configured to convert the ultrasound parameter values as determined into the image that preferably is shown to medical personnel on a screen of the system. The conversion may include a coding of the ultrasound parameter values into colors, for example, or into grey scales.

In one embodiment, with a known path length lₚ[m] per ray path p from the respective emitter element to the reflector and back to the receiver element, time of flight values Δfₚ—also referred to as delays—are calculated in function of, in this embodiment, speed of sound (SoS) increments σ_{c}—also referred to as slowness increments, per cell c, i.e.:

Δtₚ=Σc₌₁^{C}l_{p,c}σ_{c} p= . . . P, P≥C (1)

This equation (1) illustrates the time of flight values Δtₚ for a certain path p as a sum of individual speed of sound values σ_{c} per cell c, for the number of cells C that are travelled along the subject path p with the portion of the path length l_{p,c} per individual cell c.

The overall number of paths P preferably is equal to or larger than the number of cells C for a determined linear system. The system represented by equation (1) can be expressed in matrix form for all paths p representing the emitter element-receiver element combinations of the set Δt=Lσ, wherein the path lengths l_{p,c} are assembled in matrix L and represents geometric information that depends on the setup of the transducer-reflector arrangement and the size and shape of the cells, in particular their granularity/resolution.

Finding σ, which contains the slowness σ_{c} values per cell c, is the inverse problem. The resulting matrix σ hence represents the speed of sound distribution across the cells c, i.e. for the virtual cells the tissue in the subject plane is divided into, and in particular the cells c that are affected by an inclusion given that the speed of sound in such cells is different to the speed of sound in cells that cover non-tumorous tissue.

It is preferred, that for identifying a cell c in the plane, Cartesian coordinates x and y are used, preferably in an orientation with x parallel to a flat reflector, also referred to as horizontal direction, and y orthogonal thereto, also referred to as vertical direction. The indices i and j are then respectively used to enumerate cells in x and y directions.

It is preferred that both the delays Δtₚ, and slowness increments σ_{c}, as written in equation (1), represent perturbations caused by inclusions with respect to homogeneous tissue, that is, a tissue model in which no inclusions are present. Preferably, a pre-step is then used to estimate the average speed of sound v_{B} out of the measured time of flight matrix tₚ. Δtₚ then corresponds to the delays residuals after subtracting from tₚ the delays caused by the homogeneous tissue, that is,

Δtₚ=tₚ−Σ_{c=1}^{C}l_{p,c}/v_{B} (2)

Details of v_{B} calculation for a particular embodiment of the invention are later introduced in more detail in combination with FIG. 8. Quantitative speed of sound images v[m,s] are calculated from v_{B} and the slowness increments σ [s/m] for an individual cell c addressed by coordinates x and y in the plane

v(x,y)=v_{B}(1+σ(x,y))⁻¹ (3)

This inverse problem is well-posed if complete angular sets of ray paths p are available for each cell c. In other words, a set of ray paths p is available, which transverses every cell at all possible orientations [−180, 180] (o). However, this may only be achieved with high-end Ultrasound Computed Tomography (USCT) equipment containing a 360° transducer, or a rotating transducers respectively. However, with the hand-held embodiments of the apparatus described herein, only a limited set of angular directions may be covered by ray paths for a given orientation of the apparatus, and hence, only a limited set of angular ray path directions is available per cell.

This leads to two kinds of image distortion identified by the inventors:

a) Resolution loss along the missing angular directions: For instance, since ray paths parallel to the reflector are missing, a very good resolution is provided in this horizontal direction, but only a coarse resolution in vertical direction.

b) Strong streaking artifacts: These are a consequence of the steep transition at the limiting angular orientations ϕ=ϕₘₐₓ where no information exists for ϕ=ϕₘₐₓ+ε, where ε is arbitrarily small.

Therefore, it is desired to solve an incomplete reconstruction problem according to equation (2), which is inherently ill-posed. This means that the corresponding mathematical equations cannot be solved uniquely. Several potential solutions for the speed of sound matrix σ, or more general for the ultrasound parameter matrix, are possible. However, in solving equation (2) it is preferred and desired to find the solution amongst the set of possible solutions that provides the best geometric delineation of inclusions, and the best accuracy for the sound-speed values in the inclusions. However, the set of possible solutions is not to be determined: It is sufficient to determine the solution out of the set of possible solutions without the need to know these other possible solutions.

Preferably, this optimization approach is implemented by:

$$\hat{σ} = {arg min}_{σ} \{ {‖ Δ t - L σ ‖}_{2} \} \qquad ( 4 )$$

wherein it is determined, for which specific speed of sound values \hat{σ} out of the possible speed of sound values σ the error function Δt−L*σ, and preferably the second norm thereof, is minimized. Nevertheless, any norm can be used for this cost term, such as 1-norm L₁. However, in some scenarios the solution for σ in this numerically solved problem still may result in low image quality, where low resolution in vertical direction can be observed, while artifacts may impede the identification and segmentation of tumors.

Therefore, it is preferred that mathematical regularization is introduced to obtain numerically bounded solutions, which allow for satisfactory reconstructions of the position and geometry of one or more tumorous inclusions in a homogeneous tissue background.

In a first embodiment, a regularizing assumption is introduced for the smoothness of the SoS-image according to:

$${\hat{σ}}_{TV} = {arg min}_{σ} \{ {‖ Δ t - L σ ‖}_{2} + λ {‖ D σ ‖}_{n} \} \qquad ( 5 )$$

Accordingly, not only the error function Δt−L*σ is minimized but a sum of the error function and an additional term D*σ. D is a gradient matrix introducing which cells are adjacent to each other, and, correspondingly, D*σ denotes the gradient of the speed-of-sound σ of adjacent cells in the plane. The usage of the term D*σ is based on the insight that desired solutions of equation (2) show one or more closed inclusion geometries in a homogeneous tissue background. Hence, out of the set of possible SoS values solving the equation (2), those are selected, that at least in combination with minimizing the error function with piece-wise constant cell values with sudden transitions where necessary.

However, in another embodiment, D can be any other related property, such as curvature matrix (to regularize 2^{nd} order derivatives), DFT/DCT to regularize frequency components, or any wavelet transform, etc. Hence, the ultrasound parameter values can be dependent on “other linear combinations” D, such as curvature, discrete Fourier/cosine transform, wavelet transform, of the ultrasound parameter values, and thus their derivatives.

In a preferred embodiment, ∥Dσ∥ₙ minimizes a sum of horizontal and vertical gradients of the reconstructed image, and λ is a constant.

The norm n of the smoothness term D*σ critically influences the reconstruction results. For example, if the L2-norm (n=2), which is defined for an arbitrary vector x_{q} as ∥x∥₂=Σ_{q}(x_{q})², is applied to D*σ, a closed linear solution (Tikhonov regularization) of equation (5) can be found, but smooth gradients are favored with respect to sharp gradients. Large jumps in the SoS values of adjacent cells, which may contain different tissue, are penalized unnecessarily with the L2-norm, creating unrealistically smoothed results.

However, if the L1-norm n=1 is used ∥x∥₁=Σ_{q}∥x_{q}∥, which in the context of regularization is also referred to as total variation (TV) regularization or compressive sensing, sharp and smooth gradients are equally weighted, which leads to the reconstruction of piecewise homogeneous regions. With n=1, equation (5) becomes a convex problem, in particular a Second Order Cone Programming problem, which preferably is iteratively solved, with optimization methods such as the Interior Point Method and Alternating Directions Method of Multipliers (ADMM).

According to various embodiments of the present invention, the regularization term that preferably contributes to the optimization can be calculated by one norm which shows TV behavior, such as L1-norm (Eq. 6) or L2, 1-norms (Eq 7):

∥Dσ∥₁=Σ_{i,j}|σ_{i+1,j}−σ_{i,j}|+|σ_{i,j+1}−σ_{i,j}| (6)

∥Dσ∥_{2,1}=\sqrt{Σ_{i,j}|σ_{i+1,j}−σ_{i,j}|²+|σ_{i,j+1}−σ_{i,j}|²} (7)

Generally, in such grid-like organization of cells in the plane, where cells are arranged next to each other in rows in the horizontal direction as such forming columns of cells in the vertical direction, each cell has at least one neighbor in the horizontal direction and at least one neighbor in the vertical direction. Hence, such regularization term introduces directional gradients, and specifically gradients along the x-axis and another gradient along the y-axis. The indices i and j of each cell c refers to a position along the x and the y-axis respectively. The resulting SoS based image successfully filters out limited-angle artifacts and delineates closed inclusion geometries. Accordingly, the amount of information available in each angular direction is incorporated into the smoothness reconstruction.

As laid out above, missing angular orientations owed to the non-360° setup of the transducer lead to distortion in the reconstructed SoS image. However, it is known up-front which angular orientations are available for each cell, since the wave propagation/ray paths are defined by the hand-held apparatus, apart from small perturbations introduced by tumorous inclusions. Hence, it is very preferred to weight the SoS gradient contributions in different angular directions, and preferably according to the availability and/or impact of ray information in each of these directions. The resulting regularization may be referred to as “Anisotropically Weighted Spatial Regularization”. In a specific embodiment, this concept is combined with the total variation approach and is referred to, in the following, as “Anisotropically Weighted Total Variation” (AWTV).

Hence, in a most basic form for two directions such as the orthogonal directions x and y referred to above, a constant K is introduced in the regularization term as weight, which balances horizontal and vertical gradients according to the available ray information in each direction:

∥Dσ∥_{AWTV}=Σ_{i,j}κ|σ_{i+1,j}−σ_{i,j}|+(1−κ)|σ_{i,j+1}−σ_{i,j}| (8)

This can similarly be achieved for equation 7 by weighting axial components differently by parameter κ. Note that non-axis-aligned weighting can also be achieved by projecting derivative components in equations 6 and 7 onto tensors, although we herein prefer axis-aligned weighting. The weight κ can be tuned for each individual cell c. However, in a preferred embodiment, a single value κ is defined for the full image, i.e. the same weight κ is applied to all gradients of the one axis while the weight 1−κ is applied to all gradients of the other axis y. Under the assumption that κ≠0.5, the gradients along one of the directions/axis are emphasized over the gradients along the other direction/axis. In a very preferred embodiment, κ=0.9.

In another embodiment, the gradient directions used in the regularization is not limited to orthogonal directions. More than two gradient directions can be introduced in the spatial regularization term, which then may be referred to as “Multi-Angle AWTV” (MA-AWTV):

∥Dσ∥_{MA-AWTV}=Σ_{i,j}Σ_{α={α1,α2, . . . αN}}κ_{α}|D_{α}σ| (9)

where D_{α}σ=Dσ·e_{α} is the directional derivative along the unit vector with inclination α. Given the maximum available angle

ϕₘₐₓ=arc tan(0.5W/d) (10)

in the hand-held apparatus with W being the width of a linear array of transducer elements in the transducer and d being the distance between the transducer and the reflector, the gradient directions a for a total of N_{α} different directions are preferably chosen as follows:

$$\{ α \} = \{ {φ}_{i} , 180 ° - {φ}_{i} \} , \\ {φ}_{i} ∈ \{ 0 , {φ}_{max} \frac{1}{{N}_{α} / 2 - 1} , {φ}_{max} \frac{2}{{N}_{α} / 2 - 1} , … {φ}_{max} \} \qquad ( 11 )$$

and the weights κ_{α} are preferably calculated with the following algorithm:
1) Initialize weights κ_{α}={0};
2) For each wave path p crossing cell c:
   a. calculate the inclination of the wave path ϕ=±arc tan(0.5(xₑ−xᵣ)/d), where xₑ,xᵣ are the horizontal positions of the corresponding emitter element Tx and receiver element Rx of the combination, and d is the reflector depth;
   b. Increase the weight κ_{α} of the closest gradient direction {α} with respect to ϕ by the geometric overlap of path p with cell c. If |ϕ|>ϕₘₐₓ, see equation (10), do not increase κ_{α};
3) Average {κ_{α}} over all cells.

If step 3 is omitted, cell-specific κ_{α} values can also be used. In a preferred embodiment, three gradient directions are used, preferably: [0, ϕₘₐₓ, −ϕₘₐₓ], wherein 0° is defined as first direction y along the y-axis, i.e. orthogonal to the second direction along the x-axis defined by the longitudinal extension of the reflector and/or the transducer. ϕₘₐₓ is defined in equation (10).

In a preferred embodiment, equation (5) specifically is embodied as:

$${\hat{σ}}_{AWTV} = {arg min}_{σ} \{ {‖ Δ t - L σ ‖}_{2} + λ {\sum }_{i , j} \sqrt{{[ κ \vert {σ}_{i + 1 , j} - {σ}_{i , j} \vert ]}^{2} + {[ ( 1 - κ ) \vert {σ}_{i , j + 1} - {σ}_{i , j} \vert ]}^{2}} \} \qquad ( 12 )$$

In a different embodiment, equation (5) specifically is embodied as:

$${\hat{σ}}_{AWTV} = {arg min}_{σ} \{ {‖ Δ t - L σ ‖}_{1} + λ {\sum }_{i , j} κ \vert {σ}_{i + 1 , j} - {σ}_{i , j} \vert + ( 1 - κ ) \vert {σ}_{i , j + 1} - {σ}_{i , j} \vert \} \qquad ( 13 )$$

And in a further embodiment, equation (5) specifically is embodied as:

$${\hat{σ}}_{MA - AWTV} = {arg min}_{σ} \{ {‖ Δ t - L σ ‖}_{1} + λ {\sum }_{i , j} {\sum }_{α = \{ {α}_{1} , {α}_{2} … {α}_{N} \}} {κ}_{α} \vert {D}_{α} σ \vert \} \qquad ( 14 )$$

To keep the same regularization constant for equation (12) and equation (13), the weights are preferably normalized such that Σ_{α}κ_{α}=1.

It is also possible to use L2,1-norm (Eq 7) for the error function term, ∥Δt−Lσ∥_{2.1}, or any other norm that shows TV behavior.

In a preferred embodiment, the constant λ, also referred to as regularization constant, is set dependent on one or more of an image resolution and an image aspect ratio. The image aspect ratio is considered as ratio W/d with W representing the longitudinal extension of the reflector and/or transducer, and d representing the distance between the transducer and the reflector. The image resolution may be given by parameter h which denotes the height of a cell, and preferably also the width of a cell in case of square cells. In a preferred embodiment, which is later described in detail according to FIGS. 10 and 11, the constant λ in Eq 13 and Eq 14 is set as follows:

$$λ = {λ}_{ref} \frac{h}{{h}_{ref}} \sqrt{\frac{W}{d}} \qquad ( 15 )$$

wherein, in one particular example for a 128-element ultrasound array, with D=W=38E-3 m and h_{ref}=300E-6 m, the reference regularization constant is λ_{ref}=0.013 and the reference cell size h_{ref}=300E⁻³ equals the array pitch (average separation between transducer elements). An additional advantage of using total variation in the solution term is that λ does not depend on the inclusion contrast max σ_{c}, since both the error function (cost) and spatial regularization terms scale together.

In a preferred embodiment, the ultrasound parameter values are determined for several emitted ultrasound frequencies allowing to reconstruct frequency-dependence of such parameter. The measurement preferably would be repeated while setting the emitter frequency to different values in the ultrasound machine. This may allow for non-linear SoS and attenuation reconstruction. Then, different reconstructed parameters may, e.g. in their rate of change per frequency, reveal information.

In a preferred embodiment, for a specific line of cells, and most preferably for the lowest or the highest row of cells in the horizontal direction x no regularization is applied. Hence, the values for those cells are found based solely on the error function according to equation (4) when looking for the best speed of sound values for the cells of this row. By such means baseline artifacts can be released: Small DC components in the slowness distributions σ may lead to staircase artifacts in the vertical direction of the images reconstructed with equation (12) or equation (13). These artifacts can be minimized by defining a release line in the image, i.e. a row of cells, for which no smoothness regularization is applied. Typically this is performed for the lowest horizontal line (j=1). The release line accumulates DC components in σ, which are then homogeneously distributed over the image. Equation (12) is then rewritten as:

$$\begin{matrix}v = {arg min}_{σ} \{ {‖ Δ t - Lv ‖}_{1} + λ {\sum }_{i = 1 , j = 2}^{I , J} κ \vert {σ}_{i + 1 , j} - {σ}_{i , j} \vert + ( 1 - κ ) \vert {σ}_{i , j + 1} - {σ}_{i , j} \vert \} & ( 16 ) \\ {\hat{σ}}_{AWTV} = v + {( IJ )}^{- 1} {\sum }_{i = 1}^{I} {σ}_{i , 1} & \end{matrix}$$

Moreover, in the edges of the reconstructed image the gradients are not defined. Preferably, σ_{I+1,j}=0, σ_{j,J+1}=0 is set as boundary condition, which leads to a minimization of the edge slowness values, and provides a good stability in the reconstructions.

In an embodiment, prior information may be available with respect to the tissue to be examined. In such scenario, constant SoS values may be assigned for some regions of the reconstructed image. For instance, given breast tissue, a constant sound speed value each may be assigned one or more of cystic regions or fat layers. Prior information preferably referring to a region in the tissue may be introduced with the following preferred algorithm:
1) Error function ∥Δt−Lσ∥ₘ
   a) Group all σ values belonging to the same region prior information is available for region into a single value;
   b) Sum all columns of L corresponding to the grouped σ values;
2) Regularization term ∥Dσ∥ₙ
   a) Group all σ values belonging to the same region prior information is available for into a single value;
   b) Sum all columns of D corresponding to the grouped σ values.

In this embodiment, a region comprising multiple cells in the plane is treated uniformly and is assigned the known speed of sound value. Such a grouped σ region shows longer associated relative path lengths l_{p,c} in L. Consequently, an error weighting of the grouped region preferably is proportional to their surface.

Since the gradient matrix D preferably contains differences of the form [+1, −1] for adjacent cells, regularization constraints corresponding to grouped σ values will vanish. However, the edges of the prior known regions preferably will preserve the regularization constraints.

Total variation, as L1 norm used in embodiments of the reconstruction of the image, in particular performs well in reconstructing piecewise constant image regions such as inclusions, as typical for tumors and their surroundings. However, in some scenarios, it may be required to reconstruct smooth SoS regions. In these cases, the above total variation may show staircase artifacts. A possibility to alleviate these effects is to consider higher order differences in the smoothness regularization. A particular example is Total Generalized Variation:

$${‖ D σ ‖}_{TGV} = {min}_{v} \{ {‖ D σ - v ‖}_{1} + {‖ Dv - {Dv}^{T} ‖}_{1} \} \qquad ( 17 )$$

which balances between the first and second derivatives of the function. Accordingly, in another embodiment, the processor is configured to determine the speed of sound values by minimizing according to the following function:

$${‖ D σ ‖}_{TGV - AWTV} = {min}_{v} \{ {\sum }_{i , j} \vert κ ( {σ}_{i + 1 , j} - {σ}_{i , j} ) - {v}_{1} \vert + \vert ( 1 - κ ) ( {σ}_{i , j + 1} - {σ}_{i , j} ) - {v}_{2} \vert + {‖ Dv - {Dv}^{T} ‖}_{1} \} \qquad ( 18 )$$

The reconstruction of the image relies on the time of flight values tₚ identified in the measured RF traces. First, the time of flight values tₚ preferably are to be identified in the echo/RF trace received at the receiver element, prior to the tomographic reconstruction of a spatially-resolved image, in which the cumulative path perturbations are reconstructed in/projected to tissue coordinates. Such preprocessing is also referred to as delineation which is independent from the reconstruction. While image improvements including better tumor delineation and quantitative SoS reconstruction are achieved in the reconstruction step, in the delineation step is to provide suitable input data in an automatic fashion for the reconstruction step.

Typically, the RF trace received at the receiver element is a modulated ultrasound waveform with an oscillatory pressure pattern. The recorded RF trace shows multiple local maxima rather than a single pulse corresponding to the pulse triggered at the emitter element. The local maxima in addition show varying amplitudes depending on the ray path. Simply picking a maximum peak in each recorded RF trace yields incorrect time of flight values, since different peaks may be selected for different emitter element-receiver element pairs.

In a preferred embodiment of the invention, the processor is configured to simultaneously evaluate the recorded RF traces of all emitter-receiver element combinations to delineate the reflector echoes/RF traces for providing the time of flight matrix Δt which is also referred to as delay matrix. This step preferably is performed with a global optimization approach that minimizes an energy function and provides the optimum time of flight values in Δt. Regularization can be incorporated into this energy function, for instance in terms of delay continuity between adjacent emitter-receiver pairs, and/or constraints with respect to allowed reflector positions and orientations.

In one embodiment, the processor only simultaneously considers the full RF traces dataset—i.e. the digitized electrical signals over time for each receiver element. This means that the RF traces/signals are recorded prior to being analyzed given that the simultaneous analysis of all the RF traces with the same time basis is expected to result in an improved quantification of time of flight values for the delay Δt matrix.

In a preferred embodiment thereof the processor is configured to detect oscillatory patterns in the RF traces. This detection is run simultaneously on all the RF traces. The detection includes the generation of a global cost matrix C(l, t₁), which is cumulatively built along successive RF traces l (adjacent emitter-receiver pairs) for a list of N timing candidates tₗ=tₗ⁰, tₗ¹ . . . tₗ^{N} i.e., a list of possible time samples/events in the current RF trace l that may represent the pulse emitted by the emitter element, amongst which samples the best candidate is identified. Trace identifier 1 is equal to previously used trace identifier p. Preferably, a memory matrix M(l, tₗ) records discrete timing decisions for each RF trace and candidates therein. An optimum reflector timing is then found, e.g. based on Dynamic Programming (DP), by minimizing the cumulative cost, and following M(l, tₗ) backwards the optimum reflector delineation T(l):

$$\begin{matrix} ( \begin{matrix}C ( l , {t}_{l} ) \\ M ( l , {t}_{l} )\end{matrix} ) = ( \begin{matrix}{min}_{{t}_{l} - 1} \{ C ( l - 1 , {t}_{l - 1} ) + {f}_{1} ( {t}_{l} , {t}_{l - 1} ) \} + {f}_{0} ( {t}_{l} ) \\ arg {min}_{{t}_{l - 1}} \{ C ( l - 1 , {t}_{l - 1} ) + {f}_{1} ( {t}_{l} , {t}_{l - 1} ) \}\end{matrix} ) 0 & ( 19 ) \\ T ( l ) = \{ {arg min}_{{t}_{l}} C ( l , {t}_{l} ) , l = L ; M ( l + 1 , T ( l + 1 ) ) , l = 1 … L - 1 & \end{matrix}$$

with f₀ and f₁ being non-linear functions that incorporate time of flight for current tₗ and neighboring tₗ₋₁ RF traces. A general formulation of equation (19) introduces regularization into the reflector timing problem, enabling the natural incorporation of available prior information such as one or more of oscillatory pattern, smoothness, multiple echoes, path geometry into the optimization. Hence, in this embodiment, the delays of the reflector ultrasound echoes are not sequentially identified in individual RF traces corresponding to single emitter-receiver combinations, but optimized based on a global cost function, which simultaneously incorporates the information of all recorded RF traces. Such cost can also be minimized using discrete and graph-based optimization techniques well-known to those skilled in the art, such as graph-cuts, Markov-random Fields, and Conditional Random Fields. In one embodiment of the invention, the optimum reflector delineation T(l) is equal to the previously defined time-of-flight matrix tₚ. In another embodiment, the reflector geometry and the average speed of sound in tissue v_{B}, are introduced into the cost function as known parameters or optimization variables, such that the optimum reflector delineation T(l) is then equivalent to the previously defined delay residuals Δtₚ.

The described embodiments referring to the delineation step, and specifically to the identification of time of flight values from the corresponding RF traces can also be applied to arbitrary transformations of the RF traces, for instance the output of a correlator or the derivative of the signal envelope.

The ultrasound parameter that is determined per cell can be one of:
speed of (ultra)sound;
acoustic attenuation;
frequency dependent acoustic quantities;
speed of sound dispersion.

In one embodiment, the tomographic image reconstruction is based on acoustic attenuation. The acoustic attenuation α (dB/cm) describes the loss of signal amplitude due to absorption and scattering in tissue in between the transducer and the reflector. Attenuation measurements can be performed as follows with any embodiments of the apparatus and the method. In case an initial reflector delineation is defined, the delay tₚ is known for each path p, and a signal amplitude aₚ can be extracted from the signals supplied by the receiver element at tₚ.

Hence, in these embodiments, instead of the time-of-flight value, the RF wave amplitude at (around) the waveform samples corresponding to the measured delay values is identified instead which can also be corrected/scaled based on transducer and/or reflector incidence angles.

A pre-step is then used to estimate average tissue acoustic attenuation α_{B} from the measured amplitudes aₚ, based on a homogenous tissue model. The residual amplitudes in log scale log Δaₚ represent perturbations caused by inclusions with respect to homogenous tissue, and can be used to reconstruct acoustic attenuation distributions. All image reconstruction methods described in connection with speed of sound can be applied.

In a particular embodiment of the invention, which is later detailed in FIG. 8, considering the first echo transmitted between a transducer and a planar reflector, and assuming imperfect coupling between transducer elements, tissue and reflector, the average acoustic attenuation of the tissue α_{B} can be described by:

a_{e,r}=SₑSᵣR_{e,r}exp(−α_{B}d_{e,r})

d_{e,r}\sqrt{=4d²+(ξᵣ−ξₑ)²+4 sin²θ(ξᵣξₒ−d²)−2 sin(2θ)d(ξᵣ+ξₒ)} (20)

where Sₑ and Sᵣ are the sensitivities of the emitter e and receiver r elements depending on the signal coupling at their position, R_{e,r} is the reflection coefficient given e and r, α_{B} the average acoustic attenuation in tissue and d_{e,r} the path lengths as will be introduced in more detail in combination with FIG. 8. The term R_{e,r}=R^{s}_{(e+r)/2}+R^{a}_{(e-r)/2} can be split into its symmetric R^{s} component that depends on the incident reflector position-coupling term, and its asymmetric component R^{a} depending on the incident angle-reflection term. The component R^{a} can be fit or estimated from a physical model under consideration of d_{e,r}.

Accordingly, equation (20) can be rewritten in logarithmic scale:

log a_{e,r}=log Sₑ+log Sᵣ+log R^{x}_{(e+r)/2}+log R^{a}_{(e-r)/2}−α_{B}d_{e,r} log(exp(1)) (21)

Equation (21) leads to an optimization problem, which can be solved with the previously described methods. Particularly, if d_{e,r} are available, equation 21 can be cast as an overdetermined linear system of N×N equations based on N emitter-receiver pairs, and up to 4N+1 unknowns (log Sₑ, log Sᵣ, log R^{s}_{(e+r)/2}, log R^{a}_{(e-r)/2}), which can be solved, for instance, with Least-Squares. Additional simplifying assumptions can be introduced to reduce the number of unknowns. Once an estimate for the average acoustic attenuation in tissue α_{B} is obtained, residual amplitudes in log scale log Δaᵢⱼ can be used to reconstruct acoustic attenuation distributions. All image reconstruction methods described in connection with speed of sound can be applied.

In another embodiment, the tomographic image reconstruction is based on frequency dependent acoustic quantities. Given an initial reflector delineation, where the ultrasound echo delay t_{e,r} is known for each element of the emit-receive pair, the ultrasound reflector echo signal s_{e,r}(t) in function of time t can be extracted for each RF line RFₑᵣ(t):

s_{e,r}(t)=RF_{e,r}(t−t_{e,r})w(t) (22)

where w(t) is a windowing function of a given duration T, for instance a rectangular function w(t)=rect((t−T/2)/T). Other windowing functions, e.g. Hanning, Gaussian, etc. may be used in order to reduce edge discontinuities. The recorded s_{e,r}(t) are then expressed in the frequency domain f for instance, with a Fourier, cosine or wavelet transform, with separate amplitude a_{e,r}(f) and phase ϕ_{e,r}(f) components:

s_{e,r}(f)=a_{e,r}(f)exp(−îϕ_{e,r}(f)) (23)

In another embodiment, the tomographic image reconstruction is based on_speed of sound dispersion x_{c}(f), where v_{B}(f)=v_{B}(1+x_{c}(f)) can be then directly calculated from the phase ϕ_{e,r}(f) by rewriting equation (2) as:

$${t}_{e , r} ( f ) = {t}_{e , r} + \frac{{φ}_{e , r} ( f )}{2 π f} + Δ {t}_{e , r} ( f ) = {( 1 + {x}_{c} ( f ) )}^{- 1} {t}_{e , r} + Δ {t}_{e , r} ( f ) \qquad ( 24 )$$

Once the average SoS dispersion in tissue x_{c}(f) has been fitted with equation (24), the residuals of the delays Δt_{e,r}(f) are used to reconstruct frequency-dependent SoS images σ(f). All image reconstruction methods described in connection with sound of speed determination can be applied. Similarly, a frequency-dependent attenuation can be measured by replacing a_{e,r} in equation (21) with a_{e,r}(f). Then the average α_{B}(f) can be estimated. Similarly, the residuals log Δa_{e,r}(f) can be used to reconstruct frequency-dependent acoustic attenuation images. All image reconstruction methods described in connection with sound of speed determination can be applied.

In a preferred embodiment, the transducer has a linear array of transducer elements, and hence, a flat, longitudinal extension along these elements. Preferably, the reflector is a flat reflector with a longitudinal extension. However, other geometries of the transducer and/or the reflector are possible, for instance, convex implementations for one or each of. Preferably, the geometric paths between transducer pairs and reflector can be defined for such other geometries, which in general is possible for any arbitrary geometry. For this purpose, ray tracing equations or more advanced full wave simulation approaches, e.g. finite-difference time-domain simulations, can be applied.

It is preferred that two-dimensional reconstructions based on a linear array transducer, in particular which two-dimensional reconstruction of the image is in the plane defined by the transducer and the reflector. Multiple such two-dimensional measurements can be stitched together to form a three-dimensional volume. In another embodiment, two or more reflectors or array transducers, or a combination thereof can be used in order, for example, to increase field-of-view or to enrich information with more path directions for each reconstruction cell. In another embodiment, the apparatus may include a matrix transducer with a two-dimensional array of transducer elements, which allows the processing unit to reconstruct three-dimensional images, by combining emitter-receiver pair information in different planes. In a different embodiment, the two-dimensional hand-held apparatus can be used multiple times, each in a different plane, in order to generate a three-dimensional image stack. The here outlined hand-held apparatus can also be incorporated to an automated scanning system that provides three-dimensional image stack, but sequentially moving along multiple planes, which sequential movement is automatically controlled. In another embodiment, two or more reflectors (e.g. FIG. 153), or array transducers, or a combination thereof (e.g., FIG. 14), can be used in order, for example, to increase field-of-view or to enrich information with more path directions for each reconstruction cell. In a different embodiment, the two-dimensional hand-held apparatus can be used multiple times, each in a different plane, in order to generate a three-dimensional image stack. The here outlined hand-held apparatus can also be incorporated to an automated scanning system that provides three-dimensional image stack, but sequentially moving along multiple planes, which sequential movement is automatically controlled.

The present invention preferably provides an apparatus for hand-held and localized breast compression, applicable to USCT, while enabling accurately controlling the positioning and orientation between an ultrasound transducer and a reflector. Most other known breast USCT systems instead require to immerse the breast in a water tank, which adds additional complications in application, whereas the present apparatus system is hand-held, giving it flexibility in use.

Additionally, a standard ultrasound transducer can be employed which is known e.g. from conventional B-mode scanning, in contrast to customized and costly transducer mechanisms of the known systems, which then also allows a clinician to use this transducer for conventional clinical B-mode imaging, by simply decoupling other elements of the apparatus from it.

In an embodiment of the present invention, the transducer and the reflector are attached to or are integral part of a mechanical structure. The transducer and the reflector preferably are arranged opposite to each other. The mechanical structure preferably comprises a distance adjustment for enabling the sonographer to vary the distance between the transducer and the reflector. At least a part of the distance adjustment acts as indicator. Specifically, the mechanical structure comprises a first frame that the transducer is attached to, a second frame that the reflector is attached to or is integrated in or consists of, and at least a first bar both the first and the second frames are mounted to. At least one of the frames is slide-able over the first bar, e.g. by each frame providing a hole into which the bar is inserted. This first bar preferably comprises positioning means for holding the at least one frame at predefined positions such as borings in the first bar. The at least one frame comprises a pin at least partially insertable into the borings one at a time for holding the at least one frame in the predefined position at the first bar. In such embodiment, the pin preferably is mounted in the at least one frame to take a first position reaching into any of the borings, and a second position out of the borings, where the second position is required for sliding the frame between two adjacent borings of the first bar. Preferably, the pin is movable from the first position to the second position against a resilient force. The pin is preferably held into the boring by a spring mechanism adjusted so that the resilient force to achieve a second position can be achieved by hand force. Instead of pin and bores, other releasable adjusting mechanisms such as snap-fits may be used for adjusting the frame to the bar. In a different embodiment, the first bar may be a spindle of linear stage along which the first and/or the second frame may be moved, e.g. actuated via a hand wheel. Preferably, the position and/or the distance may be displayed to a user on a display assigned to the apparatus, where e.g. a position of the hand wheel is detected and converted into a distance between the transducer and the reflector. Or, a curser may be connected to the spindle and provides a distance reading to the sonographer

However, it may be preferred, for enhancing mechanical stability, that the mechanical structure comprises a second bar with the first frame being mounted to both the first and the second bar and the second frame being mounted to both the first and the second bar. Again, at least one of the frames is slidable mounted, now over both the first and the second bar. Positioning means are now provided at both the first and the second bar for holding the at least one frame at predefined positions. The positioning means preferably include borings at the predefined positions in each of the first and the second bar. The at least one frame comprises a pin at least partially insertable into the borings of the first bar and another pin at least partially insertable into the borings of the second bar for holding the at least one frame in the predefined position.

By attaching or integrating the transducer and the reflector to a hand-held operable mechanical structure, the breast is compressed only locally. The apparatus containing the mechanical structure ensures a fix relative orientation between the transducer and the reflector, provides a direct contact between the transducer and the target, e.g. the breast, preferably reduces the compression area to the active cross-section area of the ultrasound transducer, and allows for hand-held operation, which enables arbitrarily oriented scanning plane and quick adjustment of the reflector distance. Hand-held operation is standard in conventional ultrasound imaging, and is essential for sonographers during examination.

In a second embodiment, a position and/or orientation sensor is provided in the apparatus for allowing to determine a relative position and/orientation between the transducer and the reflector. Preferably, parts of the sensor are attached to both the transducer and the reflector. In one embodiment, a magnetic sensor is used, e.g. including a magnet and a sensing element for sensing a magnetic field. Other technologies, such as optical, electromagnetic, inertial positioning sensing or in general any sensor technology which records relative position and/or orientation while preserving a mostly independent movement between the transducer and the reflector is possible. Once the relative position and/or orientation of the transducer and the reflector fulfill the requirements of USCT imaging, additional misalignments may be compensated for with image processing algorithms based on the sensor information and additional features extracted from the ultrasound measurement. In this second embodiment, it is preferred that the transducer and the reflector are not mechanically connected and can be separately manipulated with respect to the breast target, e.g. with separate hands. However, for example, one or both of the transducer and the reflector may be limited in movement, and e.g. be allowed to move only in a predefined direction and/or orientation.

In a third embodiment, a single or multi-layered continuous reflector is used. A single layer may be sufficient since it may allow reflections at both a front and a back side thereof. Thin resonant reflector layers can be applied to introduce acoustic signatures in the tracked reflector signals, which can be separated from reflections observed at undesired structures e.g. within tissue or at air gaps between transducer/target breast/reflector. This allows for cancelling undesired information e.g., from the air interfaces trapped in the ultrasound gel, during an USCT image reconstruction and improves the quality of the reconstructions/imaging. Moreover, thicker reflector layers can be applied to obtain well separated ultrasonic signals from different layers. Under consideration of the layer geometry, the conjoint identification of both separated ultrasound signals provides discrimination of undesired reflective structures. Such layer surface (or thickness) can also be engineered/micro-machined, such as with a frequency ripple pattern, in order to allow for its differentiation in reflection ultrasound images. It should be noted that the reflector geometry is not limited to the presently introduced embodiments, apart from its optionally layered structure. For example, curved reflectors may be envisaged.

Preferably, at least the second frame comprising the reflector, and, if available the first frame comprising the transducer, are of a geometry that does not lead to a full breast compression. Hence, it is preferred that the frame or frames each have a width w and a length l, wherein the length l may exceed the width w, and wherein the width w of each frame may roughly correspond to the transducer's active cross-section width at least in a region designated for contacting a tissue to investigate, and e.g. be less than 2 cm, and specifically 1 cm or less. Therefore, it is avoided that the full breast is compressed between two plates as may be done in current mammography systems which translates into a painful diagnosis procedure and reduces flexibility. Instead, a relaxed pose of the patient is facilitated during inspection, and a hand-held and localized compression of the breast is achieved, while preserving accurate tracking of the reflector position and/or orientation with respect to the transducer.

Given that in all the embodiments, the relative position and/or orientation is accurately derivable and that the quality of the imaging is highly dependent on an accurate positioning and orientation between the transducer and the reflector, images of excellent quality can be achieved. Furthermore, compared to a mammography setup, the transducer is not restricted to move along a compression plate, having only access to coronal planes. Instead, the transducer is hand-operated and in direct contact with the breast, which enables flexible access to arbitrary breast positions and orientations. In the same context, small air gaps between the prior art compression plate and the breast can be avoided. These air gaps introduce strong artifacts in the USCT images. And, small-sized breasts and ultrasound imaging near the chest wall are now facilitated for accommodation compared to previous compression plate systems.

In summary, breast compression now is limited to a cross-section of the ultrasound transducer, which significantly reduces the subject pain related to the diagnosis. Arbitrary orientation and positioning of the transducer with respect to the breast is enabled, which provides similar flexibility to the sonographer for USCT compared with a conventional hand-operated B-mode transducer. Small air gaps between compression plate and breast are minimized by reducing the compression area. Moreover, remaining air inclusions can be identified and removed from the images by profiting from the layered structure of the reflector if available.

The presented invention provides a low-cost hand-held alternative to state-of-the-art high-end ultrasound tomography systems. Conventional B-mode systems can be used for USCT with a minor addition of passive mechanical components plus dedicated software. The present apparatus can be used as an add-on to conventional B-mode ultrasound equipment, particularly for breast cancer detection. However, the invention also allows for the detection and differentiation of other anomalies of the subject tissue such as lesion/fibradenoma/cysts, also giving information about size and/or depth and/or location.

Apart from breast scanning, other applications and targets may be envisaged, in which the described test geometry is applicable, e.g. in medical imaging for finger/leg/arm scanning, or in general for non-destructive testing of materials, biological or non-biological. Furthermore, other applications of a reproducible positioning of a reflector with respect to an ultrasound transducer for tomographic imaging may be found in medical imaging or even for non-destructive testing of material properties, e.g. soft or deformable solid materials, such as foams.

Other advantageous embodiments are listed in the dependent claims as well as in the description below.

### BRIEF DESCRIPTION OF THE DRAWINGS

The embodiments defined above and further embodiments, features and advantages of the present invention can also be derived from the examples to be described hereinafter and are explained with reference to the annexed drawings, wherein:

FIG. 1 illustrates a diagram of an apparatus according to an embodiment of the present invention;

FIG. 2 illustrates a diagram of an apparatus according to another embodiment of the present invention;

FIG. 3 illustrates a diagram of an apparatus according to a third embodiment of the present invention;

FIG. 4 illustrates a block diagram of a system according to an embodiment of the present invention;

FIG. 5 shows details of the embodiment of FIG. 1;

FIG. 6 shows an apparatus in a perspective view in diagram 6a), and in application to a mimic breast in diagram 6b), according to an embodiment of the invention, the apparatus of FIG. 6 preferably coinciding with the apparatus schematically shown in FIG. 1;

FIG. 7 shows an apparatus in a perspective view in an application to a mimic breast, according to an embodiment of the invention, the apparatus of FIG. 7 preferably coinciding with the apparatus schematically shown in FIG. 2;

FIG. 8 shows details of the embodiment of FIG. 3;

FIG. 9 shows a reflector arrangement of an apparatus, in an exploded view in diagram 9a), and in an assembled view in diagram 9b), according to an embodiment of the present invention, which reflector arrangement may specifically be used in the apparatus shown in FIG. 3;

FIG. 10 shows sample tomographic images reconstructed according to a data evaluation proposed according to an embodiment of the present invention;

FIG. 11 shows sample tomographic images reconstructed according to a data evaluation proposed according to an embodiment of the present invention;

FIG. 12-14 illustrate schematic views of apparati according embodiments of the present invention;

FIG. 15 illustrates a diagram of an apparatus according to an embodiment of the present invention in an application to breast inspection,

FIG. 16 illustrates in column a) different examples of artificial inclusions in a tissue, and in columns b) to f) images of simulation results achieved with a system and/or a method according to embodiments of the present invention;

FIG. 17 illustrates in graphs a.2)-a.4), b.2)-b.4) and c.2)-c.4) measuring results as used in a system according to an embodiment of the present invention.

FIG. 18 shows a schematic view of an apparatus, for which improved speed-of-sound images can be achieved applying the system and/or method according to embodiments of our invention, preferably coinciding with the methods illustrated in FIG. 16.

### DETAILED DESCRIPTION OF THE DRAWINGS

Same elements are referred to by the same reference signs across all Figures.

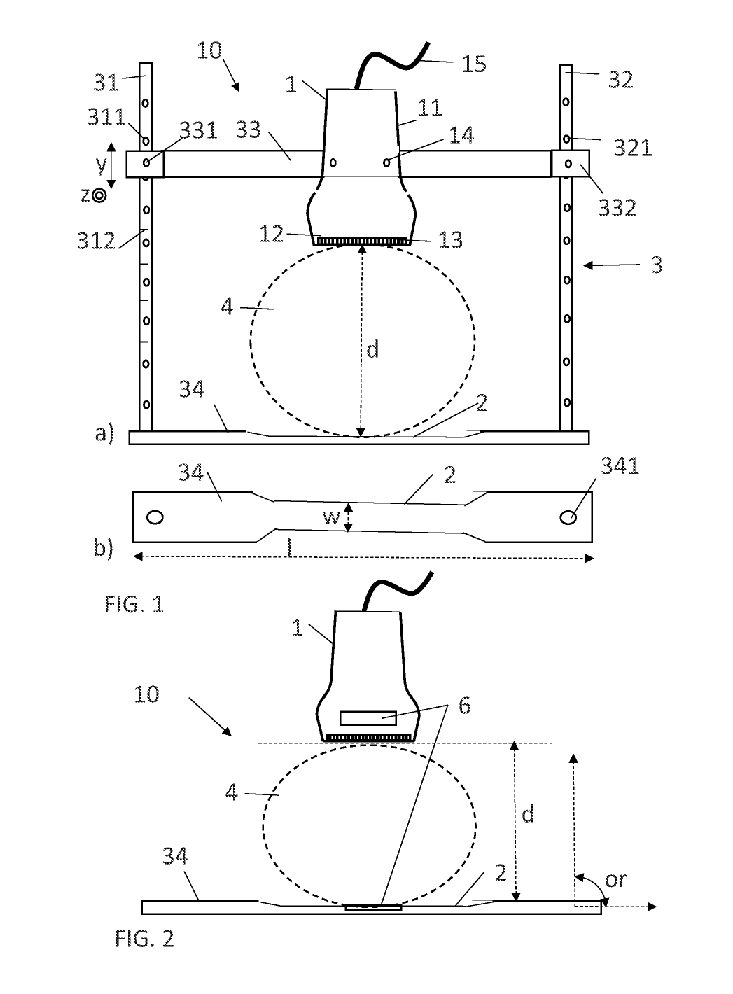

FIG. 1a) illustrates a side view of a handheld medical ultrasound apparatus 10 according to a first embodiment of the present invention. The apparatus 10 comprises an ultrasound transducer 1 and a reflector 2. The transducer 1 and the reflector 2 are arranged opposite to each other. In between, a target is arranged, the tissue of which target to be investigated is indicated by reference numeral 4, i.e. a female breast in the present example. Ultrasound waves emitted by an array of ultrasound emitters 12 travel through the tissue 4 of the breast and at least a portion thereof is reflected by the reflector 2. The transducer 12 further comprises an array of ultrasound receivers 13 for receiving reflected ultrasound waves and converting these into electrical signals. Ultrasound emitters 12 and receivers 13 can be formed by a common array as is indicated in FIG. 1.

The transducer 1 comprises a housing 11, which is fixed to a first frame 33 by means of fixing means 14 such as screws. If screw holes are not available in the transducer, the fixing means 14 can be a plastic mold that accurately reproduces the transducer geometry. The mold can be manufactured e.g. with a 3D printing device for an arbitrary commercial transducer geometry. The transducer is then inserted and fixed into the plastic mold. The transducer 1 preferably is connected via a cable 15 to a tomographic unit, preferably a conventional medical ultrasound system (not shown) and is configured to send electrical signals representing the received ultrasound waves thereto, or signals derived therefrom.

The first frame 33 is made from rigid material such as metal or plastics. The first frame 33 is slidable mounted along the y-axis over a first bar 31 and a second bar 32. The first and the second bar 31, 32 are each made from rigid material such as metal or plastics, and preferably take a cylindrical hollow shape. Each of the first and the second bar 31, 32 comprises bores 311, 321 preferably arranged equidistant as positioning means for the first frame 33. The first frame 33 comprises at each of its ends a pin 331, 332 that is capable of being at least partially inserted into one of the bores 331, 332. Each pin 331, 332 may e.g. be a bolt, a screw, or other element as long as it is insertable into a bore of the bars 31, 32. Hence, bores 311 and pin 331 together provide a means for holding a left end of the first frame 33 in a defined position, while bores 321 and pin 322 together provide a means for holding a right end of the first frame 33 in a defined position. In case the pins 331 and 332 are not inserted in any of the bores 311, 321 the first frame 33 is movable along the y-axis between two adjacent borings of each bar 31, 32. This scenario is shown in FIG. 5a) in a cut-out and with respect to pin 331. Instead, FIG. 5b) illustrates a scenario in a cut-out in which the pin 331 is inserted in one of the bores 311. The pin 331 is movable in z-direction. Preferably, the pins 331 and 332 are mounted in the first frame 33 against a resilient force, for instance, a spring mechanism, which makes the respective pin enter a bore once crossing it. For releasing the first frame 33 from a bore, the two pins 331 and 332 are lifted and slid in z-direction against the respective resilient force, e.g. manually, for allowing the first frame 33 to become movable again along the bars 31 and 32. Hence, the first frame 33 including the transducer 1 can be hand-operated vertically slid towards a second frame 34 including the reflector 2. The resilient force is sufficient high to keep the two frames 33 and 34 stable with respect to the target 4 once the position has been adjusted, but small enough to be released by hand when the frame must become movable again. Additional elements, such as a clamping ring, maybe used to stabilize the frame 33 in a defined pin position.

At their bottom end, the two bars 31 and 32 are attached to the second frame 34, preferably welded, screwed or otherwise mounted, either releasable or non-releasable. In the present example, a distance d between the transducer 1 and the reflector 2 can be adjusted by moving the first frame 33 relative to the second frame 34. In another embodiment, the second frame 34 may be additionally slidable over the two bars 31 and 32 in the same manner as is the first frame 33, e.g. by providing corresponding pins at the end of the second frame 34. In a different embodiment, the first frame 33 is fixed in its position with the bars 31 and 32, and only the second frame 34 comprising the reflector 2 is slidable over the bars 31 and 32.

Hence, frames 33 and 34 as well as bars 31 and 32 contribute to a mechanical structure 3 for holding the transducer 1 and the reflector 2, and for both allowing the distance d between the transducer 1 and the reflector 2 be varied/adjusted, and for determining a distance adjusted between the transducer 1 and the reflector 2. For supporting this purpose, one or both of the bars 31, 32 may be provided with a scale 312 allowing the sonographer to read, estimate or deduct the distance d or this distance may be read by a sensor automatically. Hence, the pin/bore-mechanism acts as a distance adjuster which on the one hand allows the fixing of a defined compression thickness by manually sliding the first frame 33 towards the second frame 34 until a release point defined by the pins entering one of the borings. The compression preferably is released by simply sliding the first frame 33 upwards. No screw loosening or tightening is necessary during this process. In one embodiment, there is not even a scale required but the sonographer can determine the distance solely by e.g. the number of free bores between the two frames 33, 34 together with the knowledge of a distance between adjacent bores.

Diagram 1b) illustrates a top view on the second frame 34 of the apparatus shown in FIG. 1a). At its ends, mounting holes 341 are provided for mounting the second frame 34 to the bars 31 and 32, e.g. for welding at these very locations. The second frame 34 has a length l and a width w, which width w is defined at a location of the second frame 34 that is expected to touch the target, i.e. the tissue 4 of breast. The second frame 34 may be of uniform width, or may be of varying width along its length l as is shown in FIG. 1b). The width w preferably roughly corresponds to the transducer's active cross-section width, which is typically less than 2 cm, preferably equal to or less than 1 cm. Hence, the second frame 34 is not a compression plate but serves for only locally compressing the breast. Preferably, the first frame 33 is of a similar width at the location of compression such that the localized compression concept is not impeded.

For taking ultrasound readings of the breast 4, the sonographer preferably moves the first frame 33 in a direction in and out of the plane of projection, thereby possibly adjusting the distance d between the transducer 1 and the reflector 2 for adapting to the shape of the breast. The sonographer may at each position record an ultrasound image which may be assembled and visualized by the tomographic unit connected to the cable 15.

The reflector 2 may be one of attached to the second frame 34, be integrated therein, or be represented by the second frame 34. E.g. in the latter case, the second frame 34 may be entirely of metal and act as a reflector 2. In a different embodiment, reflector material may be attached, e.g. be adhered to the second frame 34 which in this case may not be manufactured from an ultrasonic reflecting material but may be made e.g. from plastics.

FIG. 2 illustrates a side view of a hand-held medical ultrasound apparatus 10 according to a second embodiment of the present invention. The apparatus 10 comprises an ultrasound transducer 1 which may be identical to the transducer 1 of FIG. 1, and a reflector 2 which may be identical to the reflector of FIG. 1. The transducer 1 and the reflector 2 are arranged opposite to each other and the target to be investigated is arranged, and preferably slightly compressed in between. However, no bars are provided for providing mechanical stability and a defined distance d and/or a defined orientation between the transducer 1 and the reflector 2. However, in different embodiments, one or both or the transducer and the reflector may be mounted to allow a movement in only a defined direction or orientation. For example, the reflector may be pivot mounted at one of its end and therefore only change its position by way of rotating. In the present embodiment, a position and/or orientation sensor 6 is provided. Such sensor 6 may determine either the distance d between the transducer 1 and the reflector 2, or the orientation or there between, or preferably both. The sensor 6 may comprise elements arranged at both, the transducer 1 and the reflector 2. The sensor 6 is built based on medically approved technologies. For example, for magnetic position tracking, the sensor 6 includes a base for inducing a strong magnetic field and small receiver coils for reading the induced field. The base and the receiver coils are all connected (cabled) to the same unit to deduce position. In this case, receiver coils may be arranged at both the reflector 2 and the transducer 1. Another possibility is to use optical tracking, e.g. with infrared or visible lights, by arranging passive or active markers at both the reflector 2 and the transducer 1. Or, subject to the sensing principle, the sensor 6 may be arranged only at one of the transducer 1 and the reflector 2.

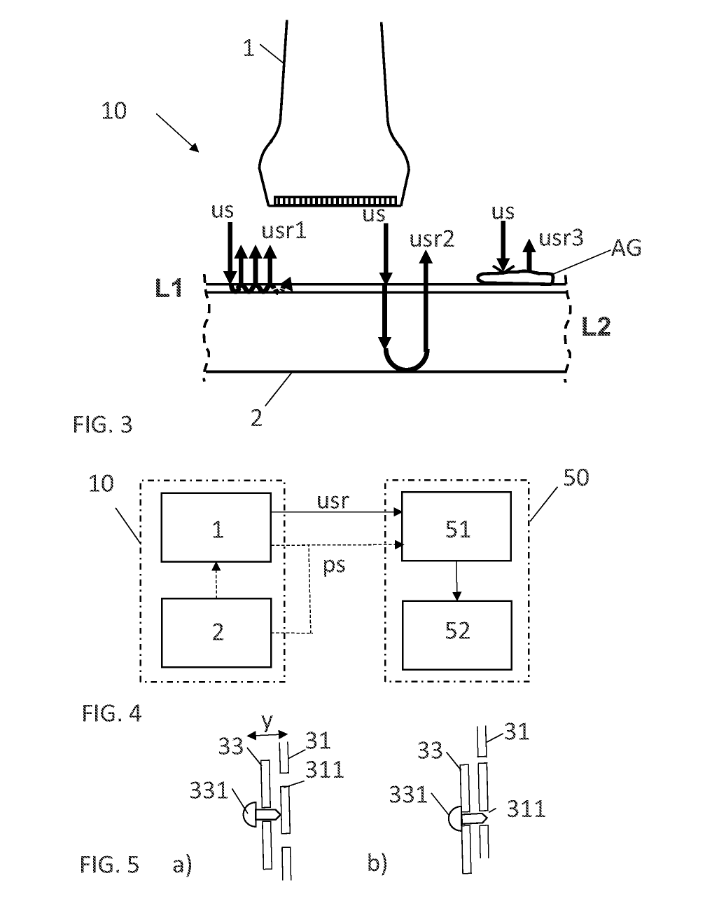

FIG. 3 illustrates a side view of a hand-held medical ultrasound apparatus 10 according to a third embodiment of the present invention. The apparatus 10 comprises an ultrasound transducer 1 which may be identical to the transducer 1 of FIG. 1, and a reflector 2. The transducer 1 and the reflector 2 are arranged opposite to each other and the target to be investigated is arranged in between (not explicitly shown in FIG. 3). Again, no bars are provided for providing mechanical stability between the transducer 1 and the reflector 2. Instead, the reflector 2 comprises a two-layered set-up including a second layer L2 with second reflection properties, and a first layer L1 on top of the second layer L2 with first reflection properties with respect to ultrasound, which second reflection properties are different to the first reflection properties. Hence, a first portion of the ultrasound us emitted is reflected by the layer L1 and is received as reflected ultrasound signal usr1 by the receiver in the transducer 1. Another portion of the ultrasound us emitted is reflected by the second layer L2 and is received as reflected ultrasound signal usr2 by the receiver in the transducer 1. The thickness of layer L1 is thin enough to induce acoustic signatures in the tracked reflector signals, for example the cancellation or enhancement of determined ultrasound frequencies. The thickness of layer L2 is large enough to obtain well separated ultrasonic signals usr2 and usr1. Both layers L1 and L2 provide complementary discrimination means to cancel reflections usr3 at undesired structures, for instance an air gap AG between tissue and reflector 2. These discrimination means can therefore be used individually, for instance, the reflector can consist only of layer L1 or L2, or combined for better discrimination. Additional layers may be added if necessary.

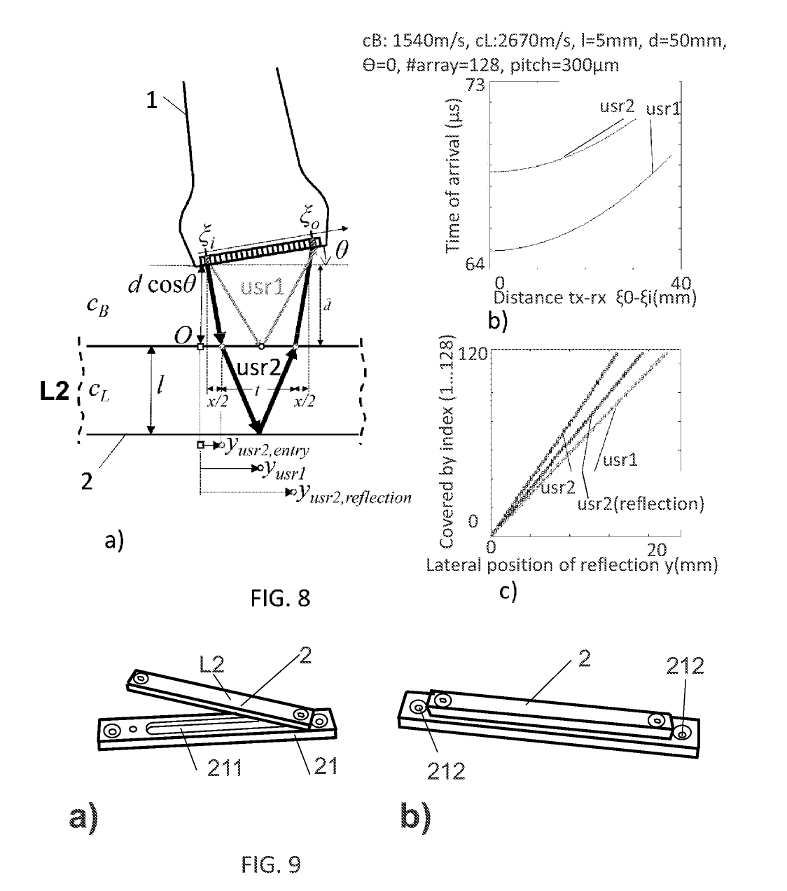

FIG. 8 illustrates a particular embodiment, in which only a single reflector layer L2 is used. An arbitrary wave propagation path between an emitter element 5i, also referred to as transmitter element, of the transducer 1 and a receiver element ξo of the transducer 1 is considered, which elements ξi, ξo may be arbitrary transducer element pairs. The transducer 1 is separated by an unknown distance d from the reflector 2, which is inclined by an unknown angle θ with respect to the transducer 1. cB is the unknown average ultrasound propagation speed in the breast tissue medium between the transducer 1 and the reflector 2 (not shown). The parameters ξi, ξo, cB are respectively equivalent to the above described parameters e, ξo and vB. The thickness l and the average ultrasound propagation speed cL in the layer L2 are known. The measured time of arrival t1, t2 of the ultrasound reflection signals at the top usr1 and bottom usr2 interfaces of the reflector 2 are functions of the unknown parameters. For reflection at the top usr1 surface, the time of arrival t1 is calculated as follows:

t₁=c_{B}⁻¹\sqrt{4d²+(ξₒ−ξᵢ)₂+4 sin²θ(ξᵢξₒ−d²)−2 sin(2θ)d(ξₒ+ξᵢ)} (25)

Note that if θ=0° the equation reduces to:

t₁²=(c_{B}⁻²)(4d²+(ξₒ−ξᵢ)²) (26)

which can be optimized for both d and c_{B} with linear least squares optimization. The former equation (25) is however not linear and must be solved with a non-linear optimization approach, preferably Nelder-Mead simplex optimization or any other appropriate method.

Hence, with equation (25), from the delays t₁ recorded from a single reflective layer, a reflector distance d, inclination θ and average ultrasound propagation speed in tissue c_{B} can be determined.

In another embodiment, equation (25) is amended by introducing an unknown time bias t_{off}, which depends on a time offset on the system lag for data acquisition, as well as on the determination which ultrasound echo feature is selected from the received signal as echoed pulse, in particular which oscillation is selected:

t₁=t_{off}+c_{B}⁻¹\sqrt{=4d²+(ξₒ−ξᵢ)²+4 sin²θ(ξᵢξₒ−d²)−2 sin(2θ)d(ξₒ+ξᵢ)} (27)

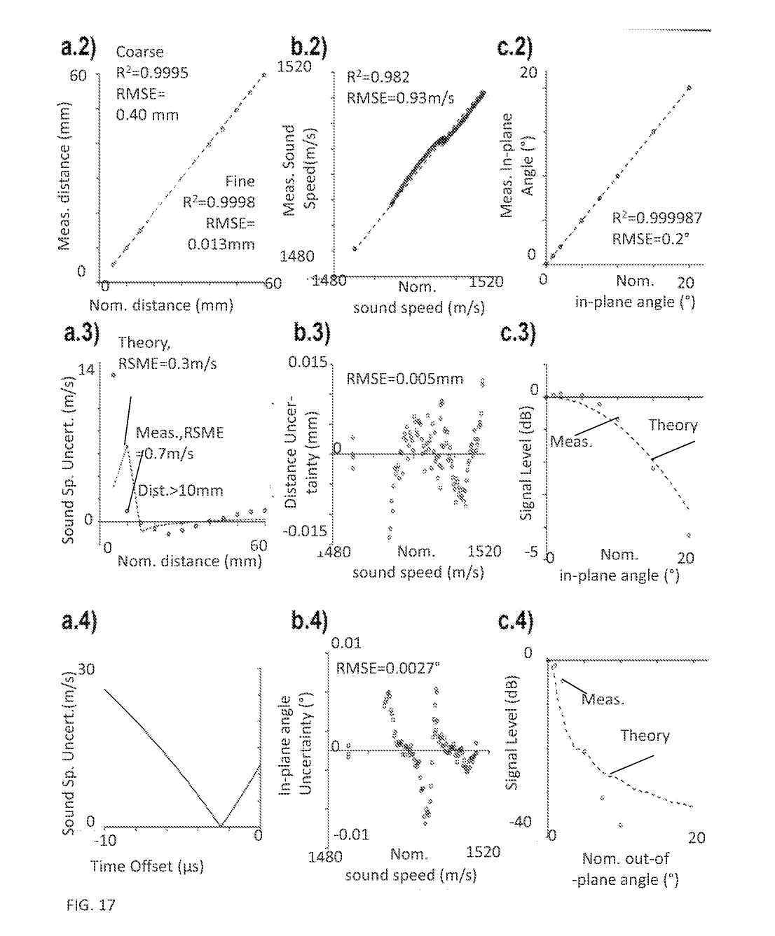

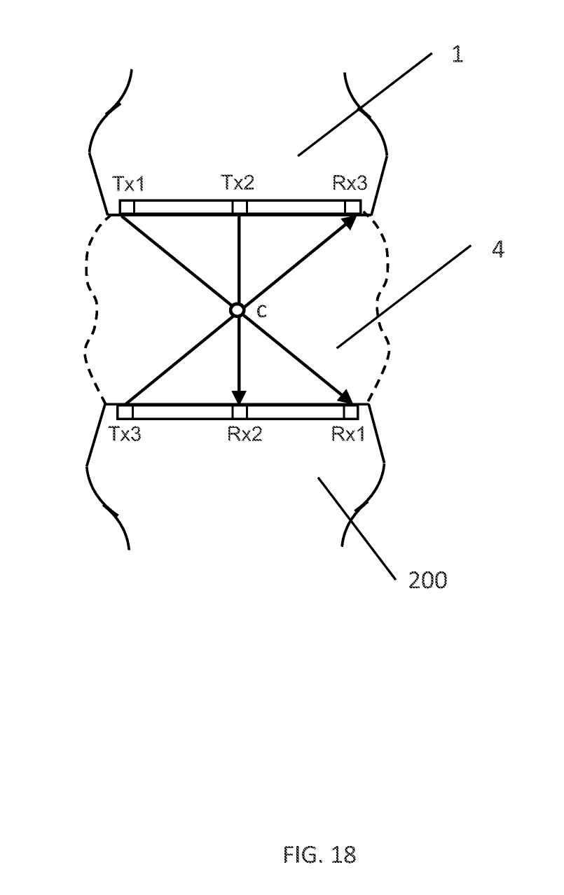

FIG. 17 shows graphs in connection with an estimation of the reflector 2 according to a preferred embodiment, and in particular its distance d from the transducer 1, and the angle θ, according to the apparatus shown in FIG. 8, from the delays of a single reflective layer tₜ according to an embodiment of the present invention.

Here, the apparatus including the transducer and the reflector was delineated in a medium, for example, distilled water medium, for which the speed-of-sound c_{B} can be precisely determined. According to the chart shown in FIG. 17 a.2), the distance d between the transducer 1 and the reflector 2 was modified. It can be derived that simultaneously estimating the time bias value t_{off}, the speed of sound c_{B}, the distance d and the angle θ leads to large errors (uncertainty >10% in c_{B}). Therefore, it is preferred to calibrate the time bias value t_{off} beforehand. The speed of sound c_{B} can be safely assumed to be constant for all distances. Therefore, a set of assumed time bias values t_{off} is assumed for fitting equation (27), and the time bias value t_{off} is selected as preferred the value of which minimizes the standard deviation of the speed of sound c₃ over all tested distances, see FIG. 17 a.4). Once the time bias value t_{off} is calibrated as described, the speed of sound c_{B} uncertainty in the function of d can be analyzed, see FIG. 17 a.3). The speed of sound c_{B} uncertainty is largest at short reflector-transducer distances, for which near field effects occur, such as between 5-10 millimeter, for example, and decreases for longer distance. These variations can be reproduced by simulating the radiated pressure fields. The distance d uncertainty for both coarse and fine calibration tests are related to the mechanical precision of the positioning frame the transducer and the reflector may be attached to, and allows for reconstruction of speed-of-sound with an accuracy <1 m/s (<0.1%).

In another step illustrated by the charts 17 b.2)-b.4), the apparatus preferably is calibrated at different speed of sound cm values for a fixed reflector position, i.e. fixed distance d and angle θ. The different speed of sound c_{B} values can be achieved by changing the temperature of the tissue/fluid between the transducer and the reflector, hence, here the temperature of the distilled water. In order to ensure a homogeneous temperature distribution, the water can be stirred with a fan during the cooling process.

Finally, and as illustrated by the charts 17 c.2)-c.4) different inclinations between the reflector 2 and the transducer 1 are tested for constant speed of sound by analyzing ultrasound reflection on a set of aluminum triangular prisms, e.g. 0°, 1°, 2°, 5°, 7.5°, 10°, 15°, and 20°, which are positioned on the reflector or separate to generate either in-plane and out-of-plane ultrasound reflections. In-plane reflections are considered to be reflections in the plane defined by the transducer and the reflector, e.g. when the reflector—or the prism in the present example—is inclined with respect to the longitudinal extension of the transducer, and hence inclined by angle θ. An out-of plane reflection is achieved, when the reflector—or the prism—in inclined with respect to a plane orthogonal to the in-plane, i.e. when the reflector is inclined around its longitudinal axis. Signal levels are measured in function of in-plane and out-of-plane inclination, see chart 18 c.3) and 18 c.4). The results are compared with simulated directivity functions for each transducer element. The in-plane inclination leads to a small signal loss in the order of 5 dB for large inclination shifts such as 20°. However, the out-of-plane inclination which is not detectable by the ultrasound transducer has a larger effect, with e.g. 20 dB signal loss for a 5° misalignment. This shows the importance of a good out-of-plane calibration, which can be achieved with the positioning frame or additional sensor means, e.g. optic or magnetic tracking sensors. If, instead of a linear transducer as in FIG. 17, a matrix transducer with a two-dimensional array of transducer elements is used, both the in-plane and out-of-plane inclination can be detected by the ultrasound transducer.

In case of a second reflective layer t₂ with respect to FIG. 8, i.e. for reflection at the bottom surface usr2, the time of arrival t2 is calculated by solving a 4^{th} degree polynomial according to equation (28):

t₂=c_{B}⁻¹\sqrt{4\hat{d}²+x₂}+c_{L}⁻¹\sqrt{4l²+(t−x)²}+c_{B}⁻¹\sqrt{[t cos θ−(ξₒ−ξᵢ)]²+[t sin θ]²}

t=(ξₒ−ξᵢ)(cos θ−sin θ)x/(2\hat{d})

p₄x⁴+p₃x³+p₂x²+p₁x+p₀=0

p₄=(c_{B}⁻²−c_{L}⁻²)[1+(ξₒ−ξᵢ)sin θ/(2\hat{d})]²

p₃=−2(ξₒ−ξᵢ)(c_{B}⁻²−c_{L}⁻²)[1+(ξₒ−ξᵢ)sin θ/(2\hat{d})] cos θ;

p₂=c_{B}⁻²[4l²+(ξₒ−ξᵢ)² cos²θ]−c_{L}⁻²[4\hat{d}²+4\hat{d} sin θ(ξₒ−ξᵢ)+(ξₒ−ξᵢ)²]

p₁=8(ξₒ−ξᵢ)c_{L}⁻²\hat{d}²[1+(ξₒ−ξᵢ)sin θ/(2\hat{d})] cos θ

p₀=−4\hat{d}²c_{L}⁻²(ξₒ−ξᵢ)² cos θ

\hat{d}=(d+tan ϕξᵢ)cos θ

where ξi is the known transmitter lateral position, ξo is the known receiver lateral position, d is the unknown distance between transducer and plate with respect to the first transducer element, θ is the unknown inclination between transducer and plate, cB is the unknown average ultrasound propagation speed in the inspected medium 4, l is the known thickness of the plate L2 and cL is the known average ultrasound propagation speed in the plate.

FIG. 8b) shows the reflection delays calculated for a preferred configuration, for the transmitter element ξi, and in which layer L2 is a 5 mm thick Plexiglas plate (cL=2670 m/s). With the chosen material parameters, a controlled and approximately constant delay between the two reflections usr1 and usr2 is achieved (˜3 ρs), which is a compromise between the time discrimination of successive ultrasound reflection signals, and the signal-to-noise ratio of the second echo usr2 which is the lower the thicker the reflector L2 is. Experimental results are shown in FIG. 11b).

FIG. 8c) illustrates the applicability of simultaneous detection of the two echoes usr1, usr2 to improve the robustness of the reflector tracker, again for the first transducer element ξi. For each lateral position y of the reflector 2 there is at least a receiver position corresponding to an ultrasound signal usr1 reflected at such position, as well as two receiver positions for ultrasound signals usr2 respectively incident and reflected at the position y. The three signals should be simultaneously detectable for the same position y and moreover provide consistent time estimates t1 and t2 according to the equations shown above. Therefore, the simultaneous consideration of multiple reflection signals in a non-linear optimization algorithm can be used to improve the accuracy of the reflection timing and to filter out reflections usr3 at undesired structures, as shown in FIG. 3. Outlier detection algorithms, for instance Random sample consensus (RANSAC) can be used to filter out such undesired structures from the measured time matrices. The equations provided above are simplified equations, which assume straight ray trajectories between transducer elements and reflector 2. In a more general implementation, these equations are refined iteratively until convergence with a full-wave solution that accounts for refraction, diffraction and scattering phenomena within the inhomogeneous tissue medium. Similarly, other wave signatures apart from the time delays (ultrasound attenuation, linear frequency response, non-linear effects) can be used for reflector tracking and tomography reconstruction with the presented embodiments.

FIG. 4 illustrates a block diagram of a system according to an embodiment of the present invention. The system comprises a portable apparatus 10 according to any one of the preceding embodiments, and a stationary tomographic unit 50 remote from the apparatus 10. A transducer 1 of the apparatus transmits electrical signals representing the reflected ultrasound waves usr to a processing unit 51 of the tomographic unit 50, where the signals usr are evaluated and preferably converted into cut view images of the target. The resulting images preferably are displayed on a display 52 of the tomographic unit 50. In case the distance and/or orientation between the transducer 1 and the reflector 2 is sensed by a corresponding sensor in the apparatus 10, it is preferred that this position information ps is transmitted to the processor unit 51, too, either by the transducer 1 or by the reflector 2, subject to the set-up of the sensor.

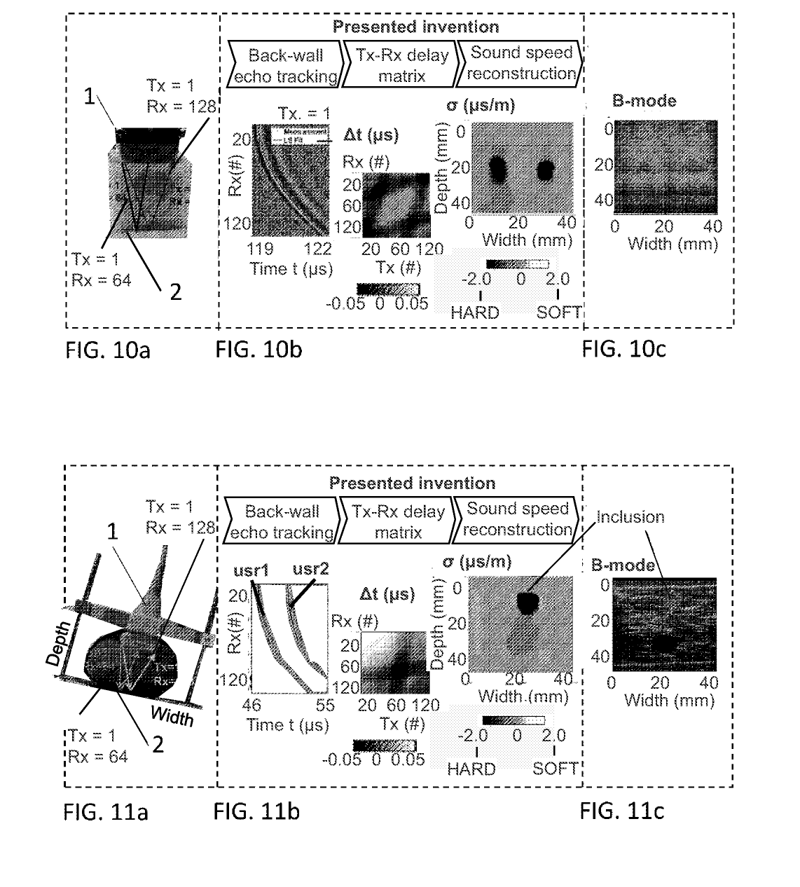

The tomographic unit 50 may in one embodiment be based on a commercial FDA-approved research ultrasound machine, e.g. a SonixTablet/SonixTouch, Ultrasonix Medical Corporation, Richmond, BC, Canada. Such machine provides a programming interface by means of which user-defined ultrasound acquisition sequences can be defined. Similar machines are available in the market from other manufacturers, e.g. Verasonics Inc., Kirkland, Wash., USA; SuperSonic, Aix-en-Provence, France. According to a preferred embodiment, for the present ultrasound tomography, the emitter and receiver array of the transducer 1 is typically operated in multi-static mode, with each element individually firing and the rest receiving. This concept is illustrated in FIG. 10a), and is equivalent to ultrasound imaging with transmitter and receiver aperture of one element. In order to improve the signal-to-noise ratio when transmitting through thick breast tissue, a larger transmitted aperture, typically 2 or 4 elements, may be used. In general, any clinically approved ultrasound machine, which allow for definition of both the transmitter and receiver aperture, may also be used for ultrasound tomography according the presented invention, as long as the reflected echoes usr1, usr2 are sampled with sufficient temporal resolution. Radiofrequency data RF is therefore in general preferred, even if B-mode images may also be used for reflector detection.

Generally, and irrespective of any of the above embodiments, in particular the distance as determined between the transducer 1 and the reflector 2 may be used in determining a speed the ultrasound travels through the target which speed may indicate tissue irregularities. And/or, the position and/or orientation between the transducer 1 and the reflector 2 may be used for identifying the relevant areas in the cut image taken by the target.

Hence, and in general, a reflector based total-variation sound-speed imaging and delineation of piecewise homogeneous inclusions in breast tissue is proposed in one embodiment, without the requirement of knowing a position of the inclusion in advance. For example, a 128-emitting and receiving element array in the transducer is operated in a multistatic mode, each element individually firing and the rest receiving. Preferably, a global optimization approach as described above (Eq 19) measures the delays of echoes reflected from the reflector behind the sample. Other algorithms based on graph theory or random Markov fields, among others, may also be used to track the delays of echoes in a continuous fashion. Non-linear optimization of the 128×128 delay matrix, for instance Nelder-Mead simplex optimization and/or RANSAC outlier filtering, provides average sound speed, plate distance and inclination, together with relative delays Δt induced by sound speed inhomogeneities. With a known geometric path-length L (i.e. the distance between the transducer and the reflector), relative slowness increments σ (low: high sound speed/hard inclusion; high: low sound speed/soft inclusion) are preferably solved from an ill-conditioned linear system Δt=Lσ. The total-variation regularization argmin_σ {∥t−Lσ∥_1+λ∥D σ∥_1}, or a variation of as described above, with D a gradient matrix, is preferably solved with convex optimization. The same equation structure or an iteratively adjusted version of it can be used for solving for other wave signatures, such as ultrasound attenuation or other linear or non-linear features.

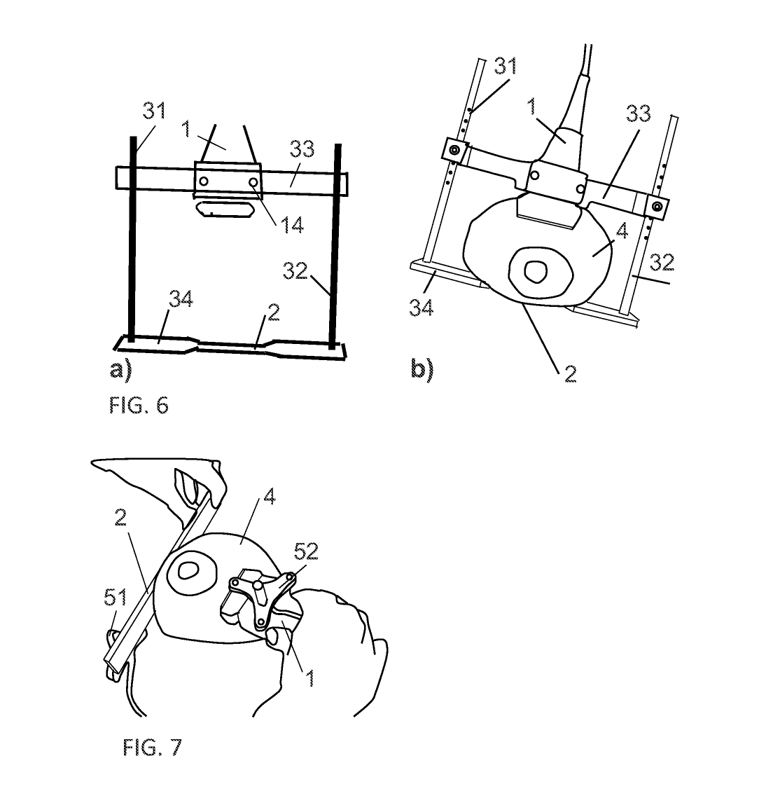

FIG. 6 shows an apparatus in a perspective view in diagram 6a), and in application to a mimic breast in diagram 6b), according to an embodiment of the invention, the apparatus of FIG. 6 preferably coinciding with the apparatus schematically shown in FIG. 1, such that the following disclosure shall in particular also be applicable to the embodiment of FIG. 1. The ultrasound transducer 1 preferably is a commercial linear array (in one embodiment presented herein: L14-5, Ultrasonix Medical Corporation, Richmond, BC, Canada). It may comprise a total of 128 transducer elements, which can alternatively act as transmitters or receivers, with a pitch between elements of 300 μm, an element elevation of 7 mm, and a total aperture of 38 mm. The transducer 1 provides two-dimensional ultrasound imaging in a plane perpendicular to the transducer elements, with the width of the image corresponding to the linear array direction, and the depth corresponding to the perpendicular direction to the transducer elements, along which ultrasound echoes are recorded in function of time, see FIG. 1 and FIG. 11. Other transducers types, for example convex array ultrasound probes or two-dimensional ultrasound arrays can be similarly used.

The reflector 2 preferably is an aluminum plate with a width w of 10 mm at the target contact region, which is arranged opposite to the transducer 1. FIG. 6b) shows the same implementation during the inspection of an ultrasound phantom (Model 059, Computerized Imaging Reference Systems, Inc (CIRS), Norfolk, Va., USA), which mimics a female breast 4. The fixing means 14 is here a plastic mold of the transducer geometry (polycarbonate) manufactured with 3D printing technology. Both first 33 and second frame 34 are made from aluminum, with second frame 34 acting simultaneously as reflector 2. The bars 31 and 32 are cylindrical and massive and are fabricated with stainless steel. The bores are 90° countersink borings machined into the bars 31,32. They are arranged equidistant as positioning reference with steps of 5 or 10 mm. The pin is a screw with a ball bearing tip, which is attached to the second frame 34 with a nut. The ball bearing is attached with a string to the screw (pressure screw) and provides an adequate resilient force for hand-held fixing and releasing the first frame 33.

FIG. 7 shows an apparatus in a perspective view in application to a mimic breast, according to an embodiment of the invention, the apparatus of FIG. 6 preferably coinciding with the apparatus schematically shown in FIG. 2, such that the following disclosure shall in particular also be applicable to the embodiment of FIG. 2. The transducer 1, reflector 2 and breast phantom 4 are the same as in FIG. 6. The transducer 1 and the reflector 2 can be moved freely and independently with separate hands. An optic sensor includes passive and active markers 51 and 52 which are attached to both transducer 1 and reflector 2, and allows real time tracking of the relative displacement and orientation between each other. The sonographer first searches for a region of interest by moving the transducer 1 along the breast 4, preferably receiving real time feedback in terms of B-mode ultrasound images on a display of an ultrasound tomography unit. Once a desired position has been identified, the reflector 2 is moved by hand until it is roughly aligned opposite to the transducer 1. Both elements are pressed slightly onto the breast, preferably with a coupling agent (e.g. water, ultrasound gel, honey, oil) in between, in order to achieve a good acoustic coupling. The optic sensor 5 provides a real time feedback on the display 52 and informs when the alignment is good enough to perform tomographic imaging. Moreover it provides geometrical referencing of the transducer 1 and reflector 2 with respect to the breast phantom, allowing the construction of volumetric breast scans by successive acquisition of ultrasound image planes at known positions and orientations.

FIG. 9 shows a reflector arrangement used in an apparatus according to an embodiment of the present invention, which reflector arrangement may specifically be used in an apparatus as shown in FIG. 3, such that the following disclosure shall in particular also be applicable to the embodiment of FIG. 3. The reflector 2 may include the specific material geometry as is calculated in connection with FIG. 8b). In contrast to FIG. 3, a single 5 mm thick, w=100 mm wide reflector layer L2 is used as reflector 2. The reflector 2 is made from Plexiglas™ (cL=2670 m/s, rhoL=1200 kg/m³), which shows a good acoustic contrast with tissue (cB=1540 m/s, rhoB=1000 kg/m³). A top surface of the layer L2 is in contact with the inspected breast tissue, providing a reflection coefficient (with normal incidence) for the reflected ultrasound signal usr1 of R=(cL*rhoL−cB*rhoB)/(cL*rhoL+cB*rhoB)=0.35 for usr1. A bottom surface of L2 is in contact with air (cAir=346 m/s, rhoAir=1.2 kg/m³), providing a strong reflection coefficient of opposite sign for the reflected ultrasound signal usr2, R=(cAir*rhoAir−cL*rhoL)/(cAir*rhoAir+cL*rhoL)=−0.9997. For a reproducible reflection behavior of usr2, the bottom surface of layer L2 preferably is kept clear. With this purpose, a plate 21 is attached below the reflector layer L2, with an engraving 211 that ensures an interface Plexiglas-air in the region of interest. The mounted reflector arrangement is shown in FIG. 9b), and may additionally be attached through screw holes 212 to e.g. the second frame 34.

FIG. 10 shows an ultrasound tomography example according to a data evaluation proposed according to an embodiment of the presented invention. A gelatin phantom 4 containing two 5 mm cylindrical inclusions is inspected with a transducer 1, with <1% ultrasound propagation speed contrast, see FIG. 11a). The inclusions do not show echo-genicity contrast with respect to the background and are therefore invisible in B-mode images, which corresponding image is shown in FIG. 11c). A large plate of a reflector 2 with a single reflecting interface is tracked. An adaptive amplitude-tracking described above successfully measures the delays of echoes reflected from the reflector 2 behind the sample, see Figure lib). Nelder-Mead simplex optimization of the 128×128 delay matrix achieves a least-square (LS) fit of the time profiles according to the multi-static wave trajectories, and provides average sound speed cₒ, plate distance dₒ and inclination θ of the reflector 2. The average sound speed cₒ has already diagnostic value, since a dense breast, which is more prone to certain pathologies, shows higher sound speed than the average breast. The proposed limited-angle reconstruction with “anisotropically weighted total-variation regularization” according to Equation 14, where three different gradient directions are calculated according to Eq 11 and the regularization parameter λ is calculated according to Eq 15, does not suffer from strong streaking artifacts as observed in previous art. On the contrary, the two inclusions are sharply and piecewise smoothly delineated in the reconstructed sound speed image, see FIG. 11b). No prior knowledge about the position or geometry of the inclusions is necessary.

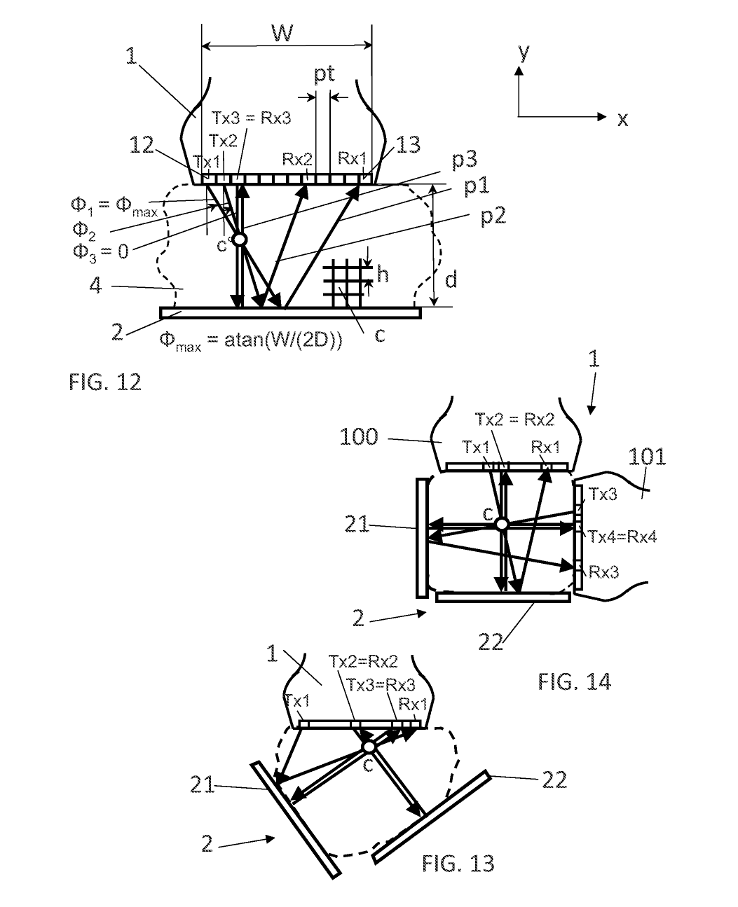

FIG. 11 demonstrates the application of the presented hand-held apparatus according to an embodiment of the present invention to cancerous mass detection. A breast phantom is investigated by an apparatus according to FIG. 6 or 1, see FIG. 12a). The reflector system illustrated in FIG. 9 is used. Two well-separated ultrasound echoes, usr1 and usr2, are obtained at the reflector 2, which allows for a robust reflection tracking as described in FIG. 8, see FIG. 12b). The second reflection signal usr2 shows opposite sign with respect to the first reflection signal usr1, due to the negative reflection coefficient in the interface Plexiglas-air. This reflection configuration allows a very robust tracking of the ultrasound signals, and achieves a high-quality time delay matrix Δt, see FIG. 12b). In comparison, it is clearly observed that measurements with wide reflector plates and a single reflector layer, as performed in the previous art mammography setups (Krueger et al. 1998, Chang et al 2007), result in significantly noisier time delay matrices Δt, due to the presence of uncontrolled air gaps and due to the presence of spurious out-of-plane propagation paths. Hence, the apparatus described in the presented invention, apart from achieving higher diagnosis flexibility and reducing diagnosis pain, provides a clear cut improvement of the quality of the recorded ultrasound signals.

The reconstructed sound speed image shows a strong and localized increase of speed of sound by about 5% (decrease of slowness), revealing a stiff inclusion at width=25 mm, depth=12 mm, see FIG. 12b). A stiff inclusion is representative of a cancerous tumor, and is therefore of highest interest in ultrasound breast diagnosis. A B-mode image of the corresponding area, see FIG. 12c) provides an indication of heterogeneity at this position, but does not provide conclusive diagnostic feedback about its nature. For example, other suspected masses between depths 20 and 30 mm in the B-mode image show much lower stiffness contrast, indicating their cystic nature, and are not revealed in the B-mode image. The “anisotropically weighted total-variation regularization approach” according to Equation 14, where three different gradient directions are calculated according to Eq 11 and the regularization parameter λ is calculated according to Eq 15, provides a piecewise smooth ultrasound image, with an excellent contrast between stiff inclusion and background. Therefore, the apparatus is capable of providing high-quality tomographic images in which tumorous inclusions can be delineated, allowing the differentiation of such inclusions from benign cystic masses.

FIG. 12 illustrates a schematic view of an apparatus according to an embodiment of the present invention, illustrating measures and dimensions useful in the reconstruction of an ultrasound image. Again, the transducer is referred to by 1 and the reflector by 2. The transducer 1 includes multiple emitters 12, also referred to by Tx1, Tx2, Tx3, etc. and multiple receivers 13, also referred to by Rx1, Rx2, Rx3, etc. wherein preferably each emitter can also be operated as a receiver, such as is indicated by “Tx2=Rx2”, and thereby representing a transducing element. Hence, the array comprises N transducer elements. A distance between adjacent transducing elements is referred to as pitch pt. An extension of the transducer elements 12, 13 in direction x, also referred to as horizontal direction x, is referred to as width W. The transducer 1 and the reflector 2 are arranged at a distance d from each other. In between the transducer 1 and the reflector 2, tissue 4 to be investigated is arranged. Preferably, the reflector 2 is flat, such that the distance d applies all across the width W.

The plane defined by the transducer 1 and the reflector 2 is made quantifiable by the Cartesian coordinates x and y, wherein y is orthogonal to x. This is the plane x, y for which an image is desired to be reconstructed. An orientation also referred to as angular direction (in this plane and is specifically related to the y orientation. Three sample ray paths p1, p2, p3 are illustrated in FIG. 12 in this plane x, y: For generating ray path p2 an ultrasound wave is fired at emitter Tx2. Its echo is received at receiver Rx2 of the same transducer element Tx2=Rx2. Here, the ray path p2 has an angular direction ϕ2=0°. For generating ray path p1 an ultrasound wave is fired at emitter Tx1. Its echo is received at receiver Rx1, wherein Tx1 and Rx1 are the two elements separated most within the row of elements. Accordingly, this corresponding ray path p1 shows the maximum angular direction (3=max possible with an apparatus of this kind. The maximum angular direction #max is determined by arc tan(W/(2d)) and hence depends on the width W of the row of transducer elements and the distance between the transducer 1 and the reflector 2. The plane x, y between the transducer 1 and the reflector 2 is virtually divided into rows and columns of cells c, oriented along the Cartesian coordinates x and y, preferably of square size h each. For each of these cells relevant to the measurement, e.g. in particular cells within the rectangle defined by width W and distance d, where tissue is present during a measurement, a speed of (ultra)sound value is determined, which may vary from cell to cell owing to tissue composition. However, in view of the setup of the apparatus and the related limited angular directions ray paths can take, each cell c is crossed only by a limited number of ray paths, i.e. only from this limited number of corresponding angular directions information can be derived for determining the speed of sound for the subject cell c. For example, and generally in such setup, a maximum angular range is given by [−ϕₘₐₓ, ϕₘₐₓ]. For a SoS image aspect ratio of 1:1, i.e. width W=depth D, the largest available angular direction is given by ϕₘₐₓ=a tan(½)=27°. The image information from angular orientations between [ϕₘₐₓ, 180° ] and [−180°, −ϕₘₐₓ] is missing. However, since ray paths, and corresponding angular directions parallel to the reflector 2, i.e. in x-direction, are missing, a very good resolution can be achieved in this horizontal direction x, but a coarse resolution only in the orthogonal vertical direction y. The solution in y direction is significantly improved if “anisotropically weighted spatial regularization”, as described above, is applied. Particularly, typical vertical inclusion elongations (>300%) with no regularization are reduced to <15% with AWSR.

In a preferred embodiment, the cell size h is chosen to be equal to the pitch pt. This measure also defines the reconstruction resolution, since it is the smallest unit in the plane for which different speed of sound values are determined that finally point to different kinds of tissue composition. Given a transducer 1 with a linear array of N elements separated by pitch pt, in operation of the system a processing unit controls and triggers a firing of ultrasound pulses at the respective emitter elements, and reads corresponding signals supplied by the receiver elements. The image aspect ratio W/d may be 1:1 with width W, and distance d, for example. Considering the problem Δt_{P×1}=L_{P×C}σ_{C×1} the number of recorded wave paths P=N². Low-populated cells, i.e. cells which are traversed by <10% of the rays that traverse the most populated cell, are preferably not reconstructed, so that in this example the number of cells C: C≈0.96N².

In a specific example of an apparatus and a system, N=128, p_{ref}=h_{ref}. For both equation (13) and equation (14), the regularization constant is λ_{ref}=0.013.

FIG. 13 shows a schematic drawing of an apparatus according to another embodiment of the present invention. In this embodiment, the reflector 2 includes two reflector portions 21 and 22 of different orientation, and in particular of orthogonal orientation in the plane (the x/y coordinates introduced in FIG. 12 shall apply to the diagrams in FIG. 13 to 15 as well). In this arrangement, the angular orientation set ϕ for the cells c can be increased.

The embodiment of FIG. 14, the transducer 1 includes two transducer portions 100 and 102, and the reflector 2 includes two reflector portions 21 and 22, opposing each other, again for increasing the angular orientation set ϕ for the cells c.

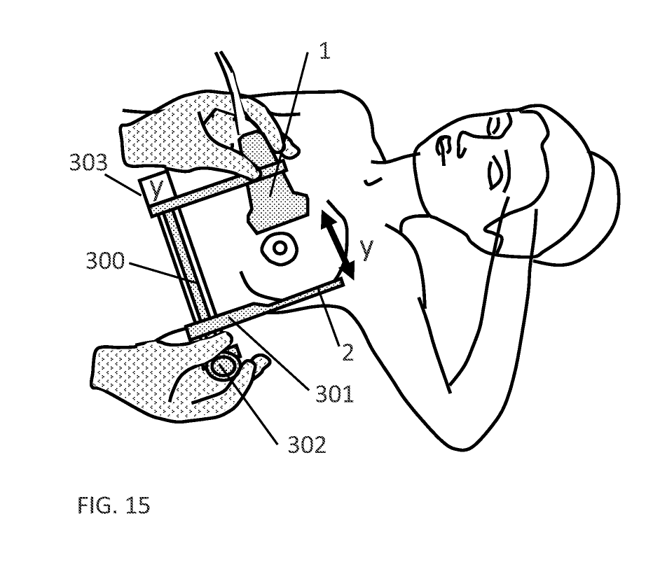

FIG. 15 illustrates a diagram of an apparatus according to an embodiment of the present invention in an application to breast inspection. In this embodiment, the first bar may be a spindle of linear stage (300) along which the first and/or the second frame (301) may be moved, e.g. actuated via a hand wheel (302). Preferably, the position and/or the distance may be displayed to a user on a display assigned to the apparatus, where e.g. a position of the hand wheel is detected and converted into a distance between the transducer and the reflector. Or, a curser (303) may be connected to the spindle and provides a distance reading to the sonographer

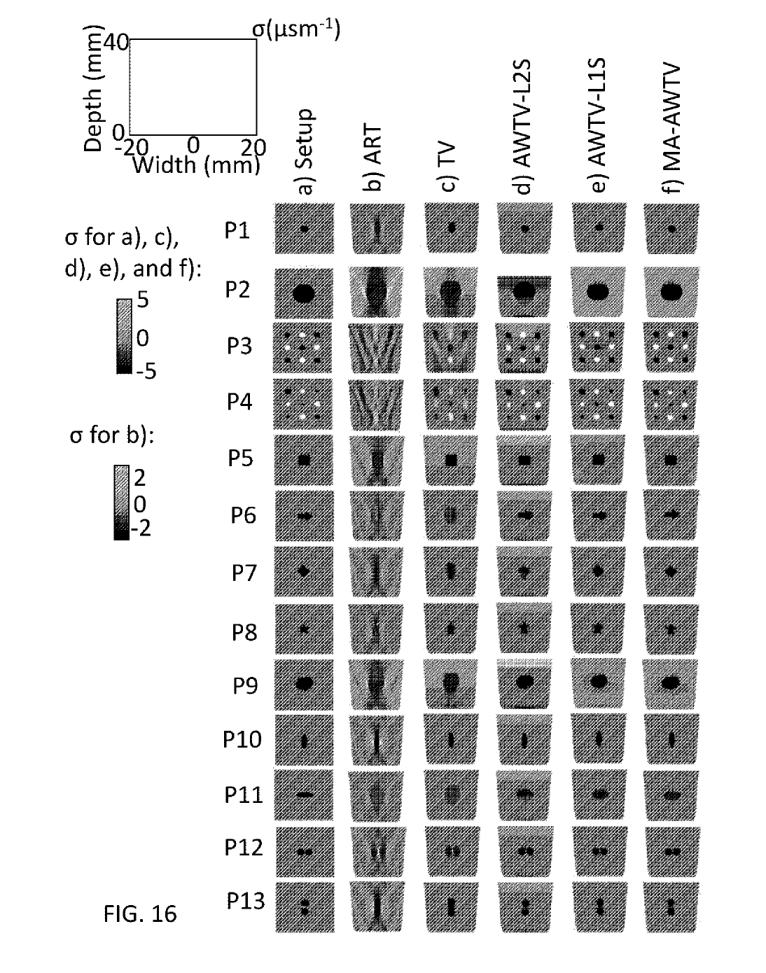

FIG. 16 illustrates in columns a) thirteen different examples of artificial inclusions (black) in a tissue (grey). Columns b) to f) show images based on simulation results of a virtual transducer extending at the top line of each sample and a virtual reflector at the bottom line of each sample, which virtual apparatus echoes the samples, and different ways of determining sound of speed values for virtual cells in each image with the respective image provided for each of the samples P1 to P13 in the respective row. In particular, the images are reconstructed with different approaches in the regularization, in particular, wherein the regularization terms are used according or included in the following equations:
b) according to the prior art implemented in “Limited-angle ultrasonic transmission tomography of the compressed female breast. Krueger et al. IEEE Ultrasonics Symposium 1998, pages 1345-1348”.
c) equation (5), “Total variation” (TV);
d) equation (6), “Anisotropically-Weighted Total Variation” (AWTV), having the L2 norm applied in the error function term as in equation (5);
e) equation (13), “Anisotropically-Weighted Total Variation (AWTV)”, having the L1 norm applied in error function term, and having release lines incorporated according to equation (16);
f) equation (14), “Multi-Angle Anisotropically-Weighted Total Variation” (MA-AWTV), with three gradient directions [0, 25°, −25°].

FIG. 18, shows an apparatus which, instead of a reflector, uses two opposed transducers (1, 201), so that specific elements of each transducer can be utilized as either transmitter or receiver elements. In this case, a return path is not required for the ultrasound waves, which minimizes signal loss and potentially allows inspecting thicker tissues. Improved speed-of-sound images can be achieved by applying the system and/or method according to embodiments of our invention, preferably coinciding with the methods illustrated in FIG. 16.

1. Hand-held medical ultrasound apparatus, comprising
an ultrasound transducer for emitting ultrasound, and
a reflector for reflecting at least a portion of the emitted ultrasound.

2. Apparatus according to claim 1, comprising
an indicator enabling the indication of a relative position and/or orientation between the transducer and the reflector.

3. Apparatus according to claim 1,
wherein the transducer and the reflector are attached to a mechanical structure opposite to each other.

4. Apparatus according to claim 3,
wherein the mechanical structure comprises a distance adjustment for varying a distance between the transducer and the reflector, at least a part of the distance adjustment acting as indicator.

5. Apparatus according to claim 3,
wherein the mechanical structure comprises a first frame the transducer is attached to, a second frame the reflector is attached to or is integrated in or consists of, and at least a first bar both the first and the second frame are mounted to, and
wherein at least one of the frames is slidable mounted over the first bar.

6. Apparatus according to claim 5,
wherein the first bar comprises positioning means for holding the at least one frame at predefined positions.

7. Apparatus according to claim 6,
wherein the positioning means includes borings at the predefined positions in the first bar,
wherein the at least one frame comprises a pin at least partially insertable into the borings for holding the at least one frame in the predefined position at the first bar.

8. Apparatus according to claim 7,
wherein the pin is mounted in the at least one frame to take a first position reaching at least partially into any of the borings, and a second position out of the borings which second position is required for sliding the frame between two adjacent borings of the first bar, and
in particular wherein the pin is movable from the first position to the second position against a resilient force.

9. Apparatus according to claim 5,
wherein the mechanical structure comprises a second bar,
wherein the first frame is mounted to both the first and the second bar,
wherein the second frame is mounted to both the first and the second bar,
wherein at least one of the frames is slidable mounted over both the first and the second bar,
wherein each of the first and the second bar comprises positioning means for holding the at least one frame at predefined positions.

10. Apparatus according to claim 9,
at the predefined positions in each of the first and the second bar, and
wherein the at least one frame comprises a pin at least partially insertable into the borings of the first bar and another pin at least partially insertable into the borings of the second bar for holding the at least one frame in the predefined position.

11. Apparatus according to claim 2,
wherein the indicator includes a position and/or an orientation sensor for determining a position and/or an orientation respectively between the transducer and the reflector, and
in particular wherein the position and/or the orientation sensor is a magnetic or optic sensor.

12. Apparatus according to claim 1,
wherein the transducer and the reflector are mechanically disconnected, and
in particular wherein the reflector is attached to or is integrated in or consist of a planar frame.

13. Apparatus according to claim 2,
wherein the indicator includes the reflector (2), which reflector comprises a single layer, or comprises more layers of different ultrasound reflecting properties.

14. Apparatus according to claim 5,
wherein the first frame and the second frame each have a width and a length, the length exceeding the width,
wherein the width of each frame is less than 2 cm at least in a region designated for contacting a tissue to investigate.

15. Medical ultrasound system, comprising
an apparatus according to claim 1,
a processor,
wherein the transducer is electrically connected to the processor, and
wherein the processor is configured to determine an ultrasound based tomographic image subject to reflected ultrasound waves received by the ultrasound transducer.

16. Medical ultrasound system according to claim 15,
wherein the ultrasound transducer comprises a set of emitter elements and a set of receiver elements,
wherein the processor is configured to, for a set of emitter element receiver element combinations, trigger the respective emitter element to emit an ultrasound wave to travel through tissue to be arranged between the transducer and the reflector, to the reflector, and from the reflector back through the tissue to the receiver element,
wherein the processor is configured to, for each of the emitter element receiver element combinations of the set, determine a time of flight value for the ultrasound wave travelling from the emitter element to the receiver element,
wherein the processor is configured to determine ultrasound parameter values of the ultrasound wave for cells in a plane defined by the transducer and the reflector, dependent on the time of flight values, and
wherein the processor is configured to convert the ultrasound parameter values into the image.

17. Medical ultrasound system according to claim 16,
wherein the processor is configured to determine the ultrasound parameter values out of a set of ultrasound parameter values dependent on gradients of ultrasound parameter values of neighboring cells.

18. Medical ultrasound system according to claim 16,
wherein the processor is configured to determine the ultrasound parameter values out of a set of ultrasound parameter values dependent on gradients of ultrasound parameter values of neighboring cells in at least two directions in the plane.

19. Medical ultrasound system according to claim 18,
wherein the processor is configured to determine the ultrasound parameter values out of the set of ultrasound parameter values dependent on gradients of ultrasound parameter values in a first direction in the plane, and dependent on a gradient of ultrasound parameter values in a second direction in the plane different to the first direction.

20. Medical ultrasound system according to claim 19,
wherein the first direction is a direction orthogonal to a longitudinal extension of the reflector and/or the transducer, and wherein the second direction is orthogonal to the first direction.

21. Medical ultrasound system according to claim 17,
wherein the processor is configured to determine the ultrasound parameter values out of the set of ultrasound parameter values dependent on gradients of the ultrasound parameter values in a first direction in the plane, dependent on gradients of ultrasound parameter values in a second direction in the plane, and dependent on gradients of ultrasound parameter values in a third direction in the plane.

22. Medical ultrasound system according to claim 21,
wherein the first direction is a direction orthogonal to a longitudinal extension of the reflector and/or the transducer,
wherein the second direction is defined by a maximum angle ϕₘₐₓ with respect to the first direction, which maximum angle is defined by ϕₘₐₓ=arc tan(W/(2*d), with W being a width of a linear array of transducer elements of the transducer and d being a distance between the transducer and the reflector,
wherein the third direction is defined by the negative maximum angle ϕₘₐₓ.

23. Medical ultrasound system according to claim 17,
wherein the processor is configured to determine the ultrasound parameter values out of a set of ultrasound parameter values dependent on weighted gradients of ultrasound parameter values of neighboring cells in at least two directions in the plane.

24. Medical ultrasound system according to claim 23,
wherein the processor is configured to apply the same weight to all gradients of the same direction, and different weights per direction.

25. Medical ultrasound system according to claim 19,
wherein the processor is configured to apply a first weight to all gradients of the first direction, and a second weight to all gradients of the second direction, wherein the first weight exceeds the second weight.

26. Medical ultrasound system according to claim 21,
wherein the processor is configured to apply a first weight to all gradients of the first direction, a second weight to all gradients of the second direction, and a third weight to all gradients of the third direction.

27. Medical ultrasound system according to claim 16,
wherein the ultrasound parameter is speed of sound,
wherein the processor is configured to determine a speed of sound value for each cell, preferably either as a single value, or in function of the frequency, or in function of any perturbation applied to the tissue.

28. Medical ultrasound system according to claim 16,
wherein the ultrasound parameter is acoustic attenuation,
wherein the processor is configured to determine an acoustic attenuation value for each cell, preferably either as a single value, or in function of the frequency, or in function of any perturbation applied to the tissue.

29. Medical ultrasound system according to claim 16,
wherein the ultrasound parameter values are identified at several emitted ultrasound frequencies allowing to reconstruct frequency-dependence of such parameter.

30. Medical ultrasound system according to claim 15,
wherein the processor is configured to determine a distance between the transducer and the reflector dependent on a time of flight value in response to triggering an ultrasound wave at the transducer.

31. Medical ultrasound system according to claim 15,
wherein the processing unit is configured to apply total-variation regularization in the calculation of tomographic ultrasound images, and in particular wherein in the total variation regularization the equation to be solved follows the form argmin_σ {∥Δt−Lσ∥_2+λ∥D σ∥_1} or any combination of such forms, where Δt is a vector of measured quantities, σ the unknown vector to be reconstructed, L a matrix geometrically calculated under consideration of the setup geometry, D a gradient matrix and λ a constant, and
in particular wherein the equation is solved with convex optimization.
